(19) United States
(12) Patent Application Publication
(10) Pub. No.: US 2025/0101064 A1
(43) Pub. Date: Mar. 27, 2025

(54) MULTIVALENT ANTIBODY RECRUITMENT MOLECULES

(71) Applicant: McMaster University, Hamilton (CA)

(72) Inventors: Anthony Rullo, Hamilton (CA); Yonghong Wan, Ancaster (CA); Benjamin Lake, Ancaster (CA)

(73) Assignee: McMaster University, Hamilton (CA)

(21) Appl. No.: 18/814,979

(22) Filed: Aug. 26, 2024

Related U.S. Application Data
(60) Provisional application No. 63/534,598, filed on Aug. 25, 2023.

(51) Int. Cl. C07K 14/00; A61K 38/00; C07K 5/02; C07K 7/06; C07K 7/56

(52) U.S. Cl. CPC C07K 14/001; C07K 5/0217; C07K 7/06; C07K 7/56; A61K 38/00

(57) ABSTRACT

The present disclosure relates generally to the field of cancer immunotherapy. More particularly, the present disclosure relates to multivalent antibody recruitment molecules and methods of treating cancer using same.

The present application claims the benefit of priority from co-pending U.S. provisional patent application No. 63/534,598 filed on Aug. 25, 2023, the contents of which are incorporated herein by reference in their entirety.

### FIELD OF THE DISCLOSURE

The present disclosure relates generally to the field of cancer immunotherapy. More particularly, the present disclosure relates to multivalent antibody recruitment molecules and methods of treating cancer using same.

### BACKGROUND OF THE DISCLOSURE

Existing cancer therapies have numerous drawbacks, including toxicity associated with chemotherapeutic drugs, development of drug resistance by cancer cells, and immune escape by cancer cells.

There is a need for new drugs and treatment modalities that forestall the development of drug resistance or immune escape by cancer cells, in order to enhance the efficacy and/or reduce the toxicity of treatment in cancer patients.

### SUMMARY OF THE DISCLOSURE

The inventors have invented multivalent antibody recruitment molecules (multivalent ARMs, also referred to as polymeric ARMs (pARMs) or multivalent immune recruiters (MIRs)), pharmaceutical compositions comprising same, and methods of treating cancer in a subject using same.

In a first aspect of the present disclosure, a compound is provided. The compound comprises:
a plurality of target binding domains (TBDs) that bind to two or more different target proteins expressed by a cancer;
at least one antibody binding domain (ABD) and/or cytotoxic agent; and
a polymer scaffold connecting the plurality of TBDs with the at least one ABD and/or cytotoxic agent.

In an embodiment of the compounds provided herein, the target proteins comprise two or more of PSMA, uPAR, HER2, EGFR, CD38, BCMA and integrins.

In an embodiment of the compounds provided herein, the target proteins comprise three or more of PSMA, uPAR, HER2, EGFR, CD38, BCMA and integrins.

In an embodiment of the compounds provided herein, the target proteins comprise four or more of PSMA, uPAR, HER2, EGFR, CD38, BCMA and integrins.

In an embodiment of the compounds provided herein, the TBDs comprise:

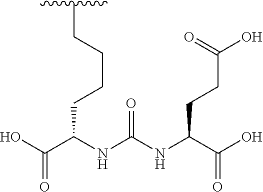 [SMILES: *CCCC[C@H](NC(=O)N[C@@H](CCC(=O)O)C(=O)O)C(=O)O]

to target PSMA;

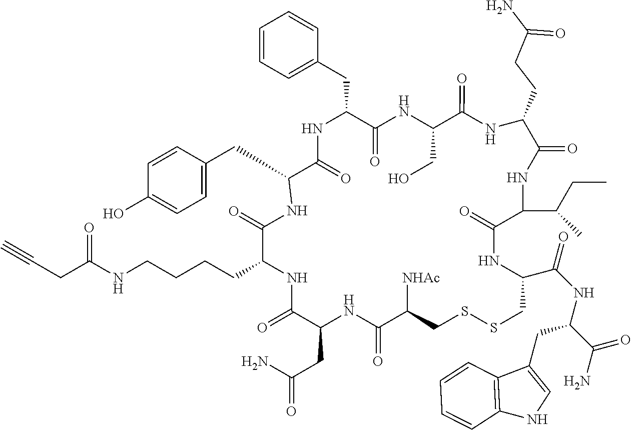 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]

or azido-K-G-S-G-G-D-Cha-F-s-r-Y-L-W-S to target uPAR, wherein Cha is cyclohexylalanine, L-amino acids are shown in upper case and D-amino acids are shown in lower case; and/or

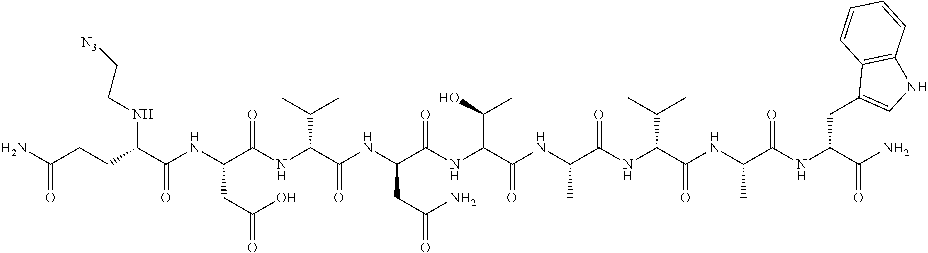 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

to target HER2.

In an embodiment of the compounds provided herein, the cancer is breast cancer, multiple myeloma, prostate cancer, or glioblastoma.

In an embodiment of the compounds provided herein, the cancer is prostate cancer and the target proteins comprise PSMA and HER2.

In an embodiment of the compounds provided herein, the cancer is prostate cancer and the target proteins comprise PSMA, HER2 and uPAR.

In an embodiment of the compounds provided herein, the cancer is glioblastoma and the target proteins comprise two or more of HER2, uPAR, PSMA and EGFR.

In an embodiment of the compounds provided herein, the cancer is multiple myeloma and the target proteins comprise CD38 and BCMA.

In an embodiment of the compounds provided herein, the cancer is breast cancer and the target proteins comprise two or more of HER2, uPAR and EGFR.

In an embodiment of the compounds provided herein, the cancer is a HER2-positive breast cancer.

In an embodiment of the compounds provided herein, the cancer is a triple negative breast cancer.

In an embodiment of the compounds provided herein, the ABD is a pan IgG binding ligand.

In an embodiment of the compounds provided herein, the ABD is a pan IgG binding ligand, and the pan IgG binding ligand is

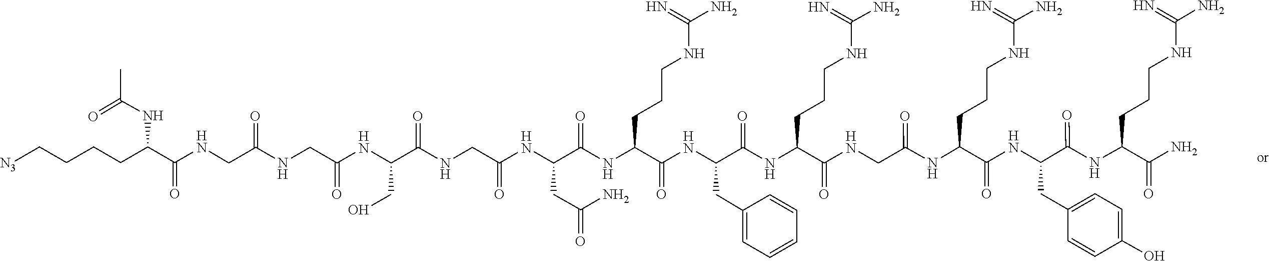 [SMILES: CC(=O)N[C@@H](CCCCN=[N+]=[N-])C(=O)NCC(=O)NCC(=O)N[C@@H](CO)C(=O)NCC(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](Cc1ccc(O)cc1)C(=O)N[C@@H](CCCNC(=N)N)C(N)=O]

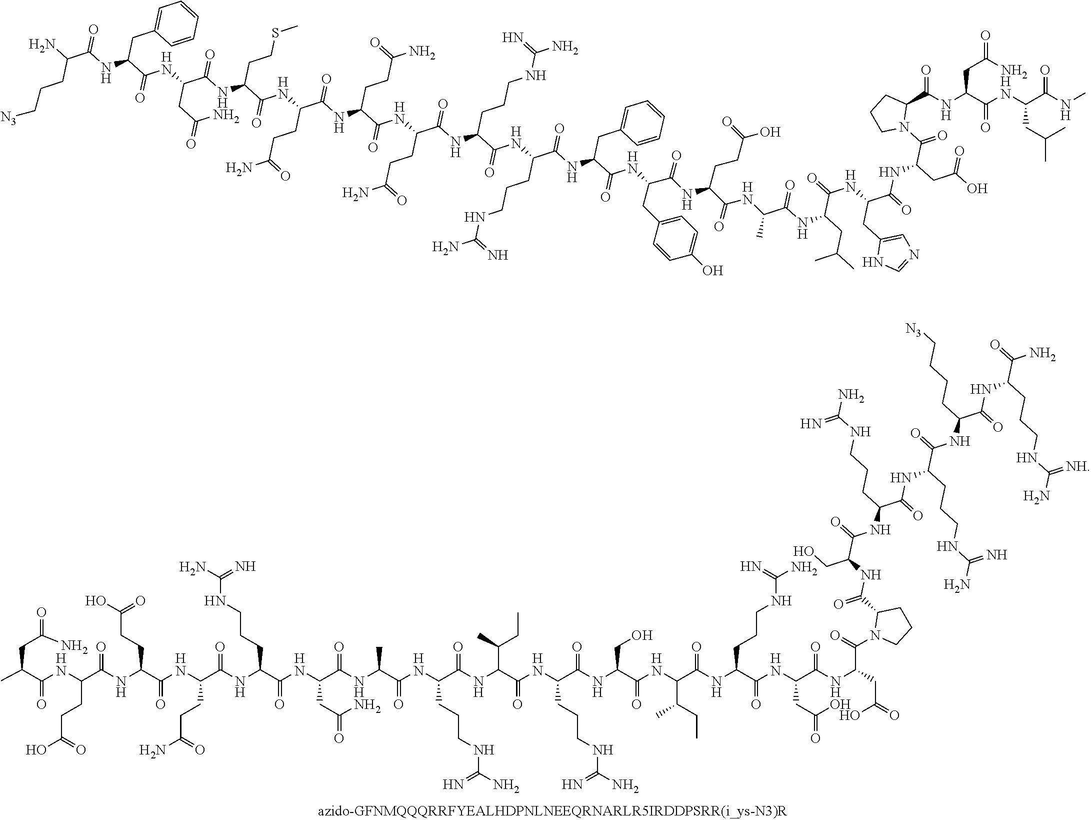

In an embodiment of the compounds provided herein, the ABD is a pan IgG binding ligand, and the pan IgG binding ligand recruits serum IgG to the cancer.

In an embodiment of the compounds provided herein, the cytotoxic agent is doxorubicin, a topoisomerase inhibitor, or a mitotic inhibitor.

In an embodiment of the compounds provided herein, the polymer scaffold comprises a Ring Opening metathesis polymer (ROMP) block co-polymer, a reversible addition-fragmentation chain transfer (RAFT) polycarboxybetaine (PCB)/methyl acrylate co-polymer or a synthetic nucleic acid biopolymer.

In an embodiment of the compounds provided herein, the ratio of TBDs: ABDs is between 1:10 and 10:1.

In an embodiment of the compounds provided herein, the ratio of TBDs: ABDs is about 2:1, about 3:2, or about 1:4.

In an embodiment of the compounds provided herein, the plurality of TBDs comprises a first TBD (TBD1) and a second TBD (TBD2).

In an embodiment of the compounds provided herein, the plurality of TBDs comprises a first TBD (TBD1) and a second TBD (TBD2), and the ratio of TBD1: TBD2 is between 1:10 and 10:1.

In an embodiment of the compounds provided herein, the plurality of TBDs comprises a first TBD (TBD1) and a second TBD (TBD2), and the compound is:

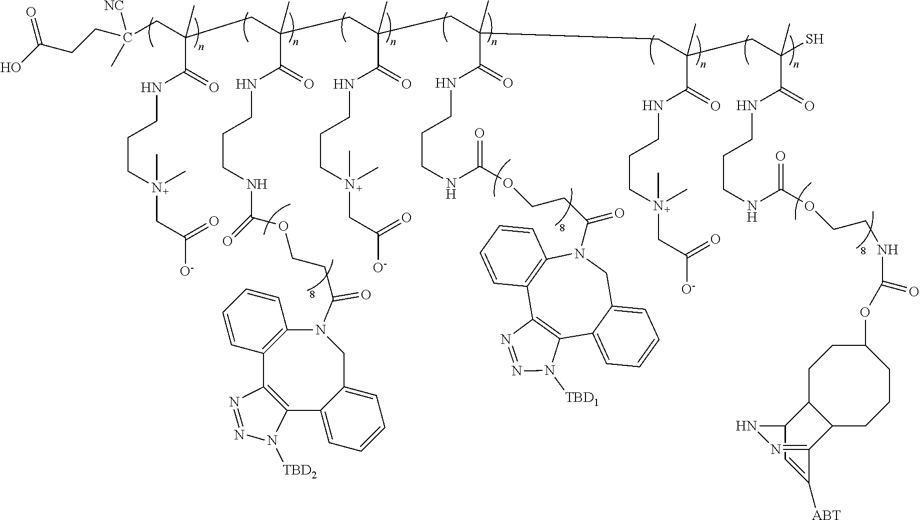

wherein n is an integer between 1 and 50,
or a pharmaceutically acceptable salt or solvate thereof.

In a second aspect of the present disclosure, a method of treating cancer in a subject is provided. The method comprises administering an effective amount of a compound to the subject, wherein the compound comprises:
a plurality of target binding domains (TBDs) that bind to two or more different target proteins expressed by the cancer;
at least one antibody binding domain (ABD) and/or cytotoxic agent; and
a polymer scaffold connecting the plurality of TBDs with the at least one ABD and/or cytotoxic agent.

In an embodiment of the method provided herein, the target proteins comprise two or more of PSMA, uPAR, HER2, EGFR, CD38, BCMA and integrins.

In an embodiment of the method provided herein, the target proteins comprise three or more of PSMA, uPAR, HER2, EGFR, CD38, BCMA and integrins.

In an embodiment of the method provided herein, the target proteins comprise four or more of PSMA, uPAR, HER2, EGFR, CD38, BCMA and integrins.

In an embodiment of the method provided herein, the TBDs comprise:

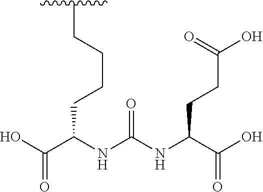 [SMILES: *CCCC[C@H](NC(=O)N[C@@H](CCC(=O)O)C(=O)O)C(=O)O]

to target PSMA;

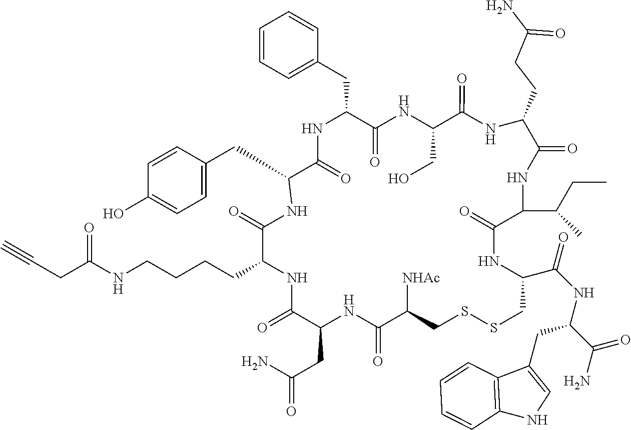 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]

or azido-K-G-S-G-G-D-Cha-F-s-r-Y-L-W-S to target uPAR, wherein Cha is cyclohexylalanine, L-amino acids are shown in upper case and D-amino acids are shown in lower case; and/or

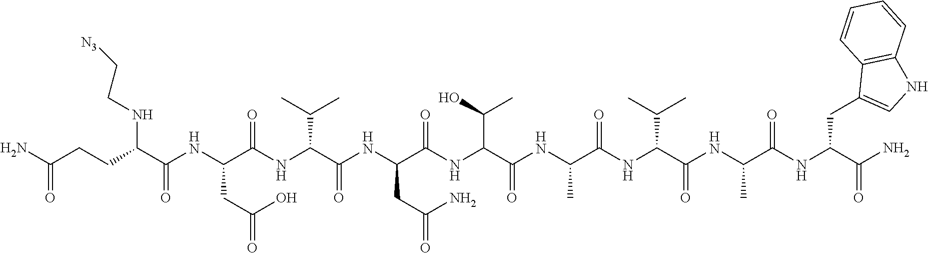 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

to target HER2.

In an embodiment of the method provided herein, wherein the cancer is breast cancer, multiple myeloma, prostate cancer, or glioblastoma.

In an embodiment of the method provided herein, the cancer is prostate cancer and the target proteins comprise PSMA and HER2.

In an embodiment of the method provided herein, the cancer is prostate cancer and the target proteins comprise PSMA, HER2 and uPAR.

In an embodiment of the method provided herein, the cancer is glioblastoma and the target proteins comprise two or more of HER2, uPAR, PSMA and EGFR.

In an embodiment of the method provided herein, the cancer is multiple myeloma and the target proteins comprise CD38 and BCMA.

In an embodiment of the method provided herein, the cancer is breast cancer and the target proteins comprise two or more of HER2, uPAR and EGFR.

In an embodiment of the method provided herein, the cancer is a HER2-positive breast cancer.

In an embodiment of the method provided herein, the cancer is a triple negative breast cancer.

In an embodiment of the method provided herein, the ABD is a pan IgG binding ligand.

In an embodiment of the method provided herein, the ABD is a pan IgG binding ligand, and the pan IgG binding ligand is

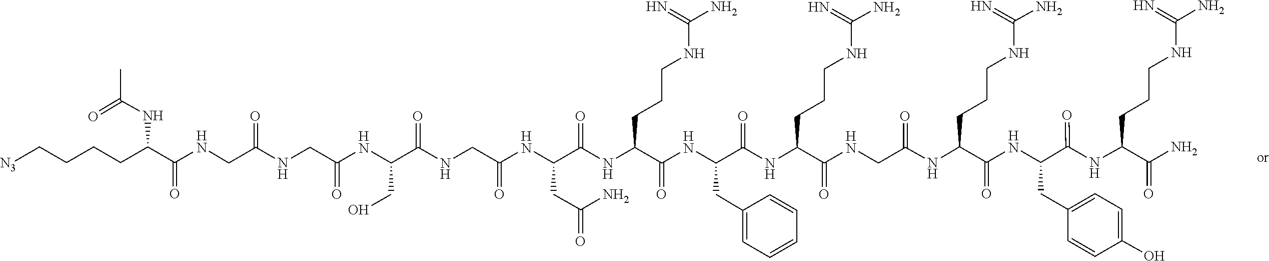 [SMILES: CC(=O)N[C@@H](CCCCN=[N+]=[N-])C(=O)NCC(=O)NCC(=O)N[C@@H](CO)C(=O)NCC(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](Cc1ccc(O)cc1)C(=O)N[C@@H](CCCNC(=N)N)C(N)=O]

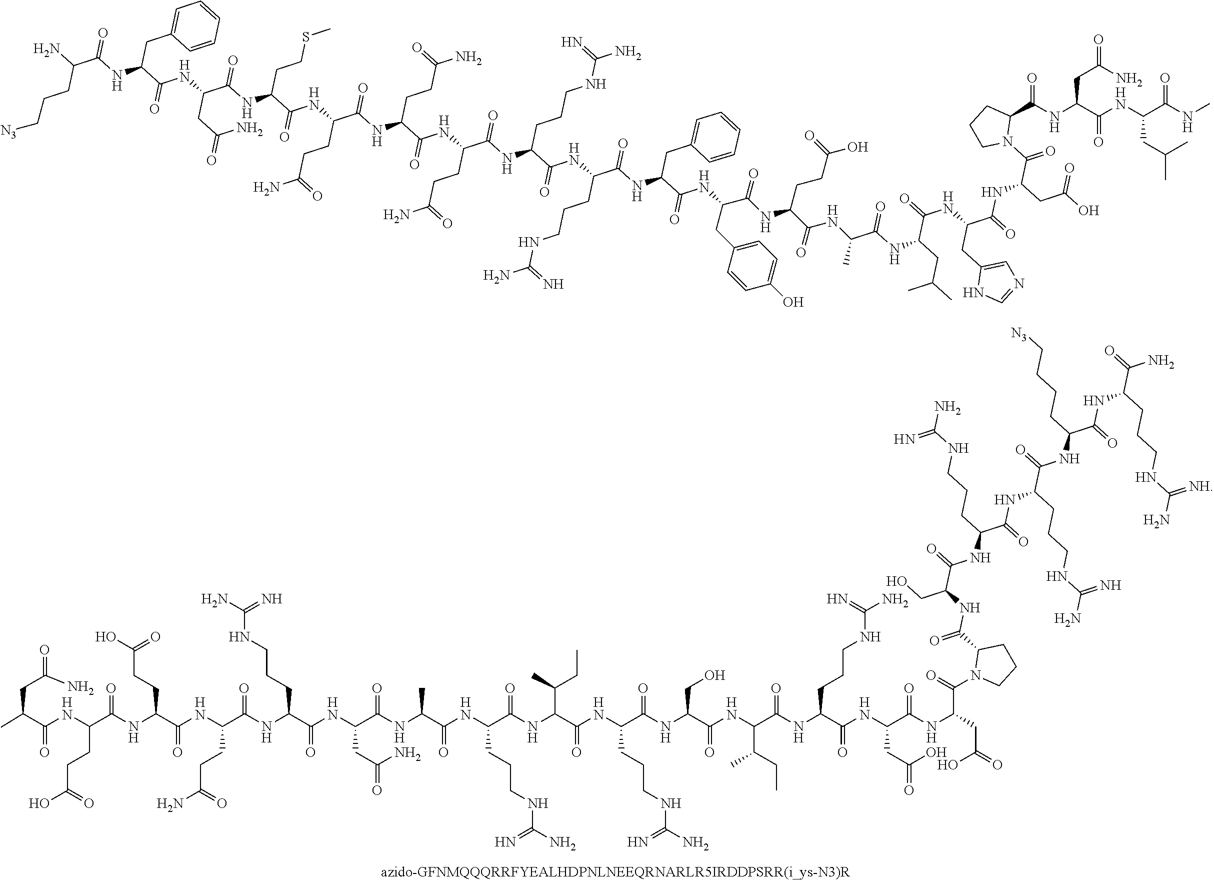

In an embodiment of the method provided herein, the ABD is a pan IgG binding ligand, and the pan IgG binding ligand recruits serum IgG to the cancer.

In an embodiment of the method provided herein, the cytotoxic agent is doxorubicin, a topoisomerase inhibitor, or a mitotic inhibitor.

In an embodiment of the method provided herein, the polymer scaffold comprises a ROMP block co-polymer, a RAFT PCB/methyl acrylate co-polymer or a synthetic nucleic acid biopolymer.

In an embodiment of the method provided herein, the ratio of TBDs: ABDs is between 1:10 and 10:1.

In an embodiment of the method provided herein, the ratio of TBDs: ABDs is about 2:1, about 3:2, or about 1:4.

In an embodiment of the method provided herein, the plurality of TBDs comprises a first TBD (TBD1) and a second TBD (TBD2).

In an embodiment of the method provided herein, the plurality of TBDs comprises a first TBD (TBD1) and a second TBD (TBD2), and the ratio of TBD1: TBD2 is between 1:10 and 10:1.

In an embodiment of the method provided herein, the plurality of TBDs comprises a first TBD (TBD1) and a second TBD (TBD2), and the compound is:

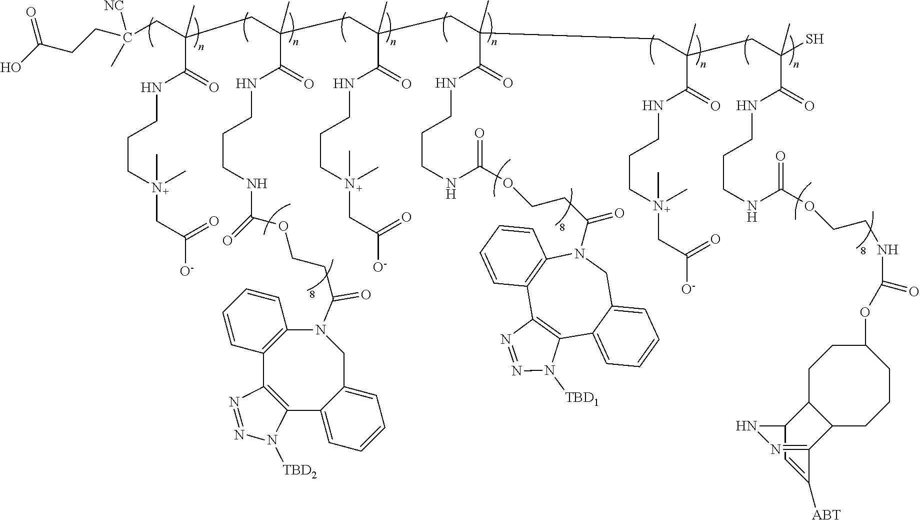

wherein n is an integer between 1 and 50,
or a pharmaceutically acceptable salt or solvate thereof.

In a third aspect of the disclosure, a pharmaceutical composition is provided. The pharmaceutical composition comprises the compound of the first aspect and a pharmaceutically acceptable carrier, diluent, or excipient.

### BRIEF DESCRIPTION OF THE DRAWINGS

In order that the subject matter of the present disclosure may be readily understood, embodiments are illustrated by way of the accompanying drawings.

FIG. 1: (A) Pictural demonstration of target-induced ARM avidity, as well as MIR inherent ABD/TBD avidity targeting. (B) Synthetic route for modular construction of MIRs.

FIG. 2: BLI to measure MIR mediated antibody recruitment to a PSMA-coated surface. (A) BLI analysis of construct recruitment to a PSMA-coated probe and subsequent antibody recruitment. (B)/(C) Pictorial illustrations of each phase for MIRs (B) or monomeric ARM (C).

FIG. 3: Ability of MIRs to bind with avidity and recruit antibody to a PSMA-coated cellular surface. (A) Competition experiment with fluorescent-ABD ARM containing a GUL TBD. (B) Cell surface residency of constructs vs time post cell wash.

FIG. 4: Capacity of MIRs to recruit anti-DNP antibody to model cancer cell lines varying in PSMA-valency.

FIG. 5: Ability of constructs to illicit antibody dependent cellular phagocytosis (ADCP) of relevant PSMA expressing cancer line C4-2. (A) Cartoon representation of MIR induced ADCP of target cells. (B) ADCP as measured by flow cytometry.

FIG. 6: Synthesis of multivalent ARMs. “n” is an integer between 1 and 50.

FIGS. 7A and 7B: Synthesis of multivalent ARMs having two or more different TBDs. FIG. 7A is a scheme showing the conjugation of NHS-linker substituted DBCO and TCO handles to a polymer scaffold. FIG. 7B is a scheme showing the click chemistry-mediated conjugation (i) of the target binding domains (TBD1 and TBD2) to DBCO, and (ii) of the antibody binding terminus (ABT) or cytotoxic payload to TCO. “n” is an integer between 1:50.

Other features and advantages of the present disclosure will become more apparent from the following detailed description and from the exemplary embodiments.

### DETAILED DESCRIPTION OF THE DISCLOSURE

### Definitions

The terminology used herein is for the purpose of describing particular embodiments only and is not intended to be limiting of example embodiments of the invention. Unless defined otherwise, all technical and scientific terms used herein generally have the same meaning as commonly understood by those of ordinary skill in the art. Generally, nomenclatures used in connection with synthetic chemistry, organic chemistry, biochemistry, molecular biology, cell and tissue culture, immunology, genetics, etc. described herein are those well-known and commonly used in the art. Nevertheless, definitions of selected terms are provided for clarity and consistency.

The following list includes abbreviations used repeatedly throughout the present disclosure:
ABD: antibody-binding domain (of antibody recruitment molecule) (also referred to as antibody binding terminus (ABT))
ADCC: antibody-dependent cellular cytotoxicity
ADCP: antibody-dependent cellular phagocytosis
ARM: antibody recruitment molecule (also referred to as antibody engager (AE) molecule or immune proximity inducing molecule)
BCMA: B-cell maturation antigen (also known as BCM, TNFRSF17, CD269)
BLI: bio-layer interferometry
Boc: tert-butyloxycarbonyl protecting group
CD38: cluster of differentiation 38 (also known as ADPRC1)
EGFR: epidermal growth factor receptor (also known as ERBB, ERBB1, HER1)
Fab: antigen-binding fragment region of an antibody
Fc: fragment crystallizable region of an antibody
GUL: glutamate urea lysine (also abbreviated as GU)
HER2: human epidermal growth factor receptor 2 (also known as HER2/neu, ERBB2, CD340)
IEDDA: inverse electron demand Diels-Alder (a type of “click” chemistry reaction)
IgG: immunoglobulin G
lgM: immunoglobulin M
NHS: N-hydroxy succinimide
PARM: polymeric antibody recruitment molecule (also referred to as a multivalent ARM, multispecific ARM or multivalent immune recruiter (MIR))
PSMA: prostate-specific membrane antigen (also known as PSM, GCP2, FOLH1, NAALAD1)
RAFT: reversible addition-fragmentation chain-transfer polymerization
ROMP: ring-opening metathesis polymerization
SPAAC: strain-promoted azide-alkyne cycloaddition (a type of “click” chemistry reaction)
TBD: target-binding domain (of antibody recruitment molecule)
uPAR: urokinase-type plasminogen activator receptor (also known as CD87)

As used herein, the singular forms “a,” “an,” and “the,” are intended to include the plural forms as well, unless the context clearly indicates otherwise.

The phrase “and/or” should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the phrase “one or more,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “one or more” refers, whether related or unrelated to those elements specifically identified. Thus, as a non-limiting example, “one or more of A and B” (or, equivalently, “one or more of A or B,” or, equivalently “one or more of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The term “consisting of” and its derivatives, as used herein, are intended to be closed terms that specify the presence of stated features, integers, steps, operations, elements, and/or components, and exclude the presence or addition of one or more other features, integers, steps, operations, elements and/or components.

The term “isolated molecule” (where the molecule is, for example, a small molecule, a polypeptide, a polynucleotide, or an antibody or fragment thereof) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

As used herein, an “antibody” is an immunoglobulin molecule capable of specific binding to a target, such as an antibody binding domain of an ARM, carbohydrate, polynucleotide, lipid, polypeptide, etc., through at least one antigen recognition site, located in the variable region of the immunoglobulin molecule. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. In an embodiment, the antibody is a human antibody. In an embodiment, the antibody is a serum antibody.

As used herein, the term “cytotoxic agent” or “cytotoxic payload” refers to any small molecule compound that elicits toxicity toward a cancer cell or tissue, and is suitable for conjugation to the multivalent ARM molecules disclosed herein. Cytotoxic agents according to the present disclosure include, but are not limited to, doxorubicin, topoisomerase inhibitors, mitotic inhibitors, and any drugs or chemotherapies that are used in antibody-drug conjugates. Cytotoxic agents suitable for use with the multivalent ARM compounds of the present disclosure may be determined by a person of skill in the art.

As used herein, the term “polymer scaffold” refers to a polymeric linker or backbone through which the plurality of TBDs and at least one ABD are connected. The polymer scaffold may comprise any natural or synthetic polymer or co-polymer that is chemically suitable for conjugation to the TBD and the ABD, and is pharmaceutically suitable for administration to a subject, such as a human subject. The selection of a polymer scaffold suitable for use with the multivalent ARM compounds of the present disclosure may be determined by a person of skill in the art. In certain embodiments, the polymer scaffold comprises a ROMP block co-polymer, a RAFT PCB/methyl acrylate co-polymer or a synthetic nucleic acid biopolymer.

As used herein, “substantially pure” means an object species, for example, an ARM compound or a component of an ARM compound of the disclosure, is the predominant species present (i.e., on a molar basis it is more abundant than any other individual species in the composition), and preferably a substantially purified fraction is a composition wherein the object species (e.g., a protein or a polypeptide) comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “treatment,” “treat,” “treating” or “amelioration” as used herein is an approach for obtaining beneficial or desired clinical results. For purposes of this disclosure, beneficial or desired clinical results include, but are not limited to, one or more of the following: decreased extent of damage from a disease, condition, or disorder, decreased duration of a disease, condition, or disorder, reduction in the number, extent, or duration of symptoms related to a disease, condition, or disorder, an increase in the period of time prior to a relapse of a disease, condition, or disorder in a subject, and/or an increase in the disease-free or overall survival rate of a subject having a disease, condition, or disorder. The term includes the administration of the compounds, agents, drugs or pharmaceutical compositions of the present disclosure to prevent or delay the onset of one or more symptoms, complications, or biochemical indicia of a disease or condition; lessening or improving one or more symptoms; shortening or reduction in duration of a symptom; arresting or inhibiting further development of a disease, condition, or disorder; or decreasing the toxicity of a therapy. Treatment may be prophylactic (to prevent or delay the onset of a disease, condition, or disorder, or to prevent the manifestation of clinical or subclinical symptoms thereof) or therapeutic suppression or alleviation of symptoms after the manifestation of the disease, condition, or disorder. Treatment may also be maintenance therapy to decrease the chances that a disease, condition, or disorder will reoccur or to delay recurrence of a disease, condition, or disorder. The beneficial result may be an increase or decrease (as appropriate) of 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% relative to an appropriate control, for example, a subject that did not receive the therapy.

In an embodiment of the present disclosure, the disease, disorder or condition treatable by the compounds, pharmaceutical compositions and methods provided herein is cancer. In an embodiment, the cancer is one that is impacted or treatable by immunotherapy, either alone or in combination with one or more other therapies, such as chemotherapy. In an embodiment, the cancer is one that is impacted or treatable by activation of endogenous immune cells. In an embodiment, the cancer is one that is impacted or treatable by stimulating an immune response to tumor cells. In an embodiment, the cancer is one that is impacted or treatable by provoking phagocytosis of tumor cells.

As used herein, the term “cancer” refers to any pre-neoplastic or neoplastic cell, tissue, organ, or organ system. In various embodiments, the cancer is a cancer cell, a solid tumor or a blood cancer.

In certain embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Triple Negative Breast Cancer; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioblastoma Multiforme; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kdposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kdposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In an embodiment, the cancer is prostate cancer, breast cancer, multiple myeloma or glioblastoma. In an embodiment, the cancer is prostate cancer. In an embodiment, the cancer is breast cancer. In an embodiment, the cancer is triple negative breast cancer. In an embodiment, the cancer is HER2-positive breast cancer. In an embodiment, the cancer is multiple myeloma. In an embodiment, the cancer is glioblastoma multiforme.

A “subject” is a vertebrate, preferably a mammal (e.g., a non-human mammal), and still more preferably a human. In an embodiment, the subject may be any human patient. In an embodiment, the subject may be limited to one or more patient subpopulations, such as, but not limited to, a female patient, a male patient, a geriatric patient, a pediatric patient, a patient with specific comorbidities and/or a patient with one or more genetic predispositions to hereditary cancer(s).

The term “administering” or “administration” as used herein refers to the placement of an agent, a drug, a compound, or a pharmaceutical composition as disclosed herein into a subject by a method or route which results in at least partial delivery of the composition to a desired site. The compounds and pharmaceutical compositions disclosed herein can be administered by any appropriate route which results in an effective treatment in the subject. Possible routes of administration of the compounds and pharmaceutical compositions disclosed herein include, but are not limited to, intravenous, intraperitoneal, intramuscular, subcutaneous, transdermal, oral, buccal, sublingual, intranasal, or rectal routes of administration, or a combination thereof.

The term “effective amount” or “therapeutically effective amount” as used herein is an amount sufficient to affect any one or more beneficial or desired results. In more specific aspects, an effective amount may alleviate or ameliorate one or more symptoms of cancer, decrease the duration of time that one or more symptoms of cancer are present in a subject, reduce the size of a tumor in a subject, eliminate all detectable levels of a tumor in a subject, increase the period of time prior to a relapse of cancer in a subject, and/or increase the disease-free or overall survival rate of a subject having cancer. For prophylactic use, beneficial or desired results may include eliminating or reducing the risk, lessening the severity, or delaying the onset of cancer or a particular stage/grade of the cancer, including biochemical and/or histological symptoms of the cancer, its complications and intermediate pathological phenotypes presenting during development of the cancer. For therapeutic use, beneficial or desired results may include clinical results such as reducing one or more symptoms of cancer; decreasing the dose or length of administration of other medications required to treat the cancer; enhancing the effect and/or reducing the toxicity of another medication; delaying the progression of the cancer a subject, decreasing the duration of time that one or more symptoms of cancer are present in a subject, increasing the period of time prior to a relapse of cancer in a subject, and/or increasing the disease-free or overall survival rate of a subject having cancer. An effective amount can be administered in one or more than one dose, round of administration, or course of treatment.

For purposes of this disclosure, an effective dosage of a compound or a pharmaceutical composition is an amount sufficient to accomplish prophylactic or therapeutic treatment either directly or indirectly. As is understood in the clinical context, an effective dosage of a compound, or a pharmaceutical composition may or may not be achieved in conjunction with another agent, drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. The amount may vary from one subject to another and may depend upon one or more factors, such as, for example, subject gender, age, body weight, subject's health history, and/or the underlying cause of the disease, condition, or disorder to be prevented, inhibited and/or treated.

As used herein “pharmaceutically acceptable” means compatible with the treatment, diagnosis or analysis of subjects.

The term “pharmaceutically acceptable carrier, diluent, or excipient” as used herein includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. In some embodiments, diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 20th Ed. Mack Publishing, 2000).

The term “epitope” refers to the area or region of an antigen to which an antibody specifically binds, e.g., an area or region comprising a contact residue that interacts with the antibody. Thus, the term “epitope” refers to that portion of a molecule (e.g., an ABD of an ARM) capable of being recognized by and bound by an antibody at one or more of the antibody's antigen-binding regions. Typically, an epitope is defined in the context of a molecular interaction between an antibody, or antigen-binding fragment thereof, and its corresponding antigen. Epitopes often consist of a surface grouping of molecules such as amino acids or sugar side chains and have specific three-dimensional structural characteristics as well as specific charge characteristics.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids, The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Also included within the definition are polypeptides, oligopeptides, peptides and proteins having amino acid sequence identity to a given polypeptide, oligopeptide, peptide or protein. The percent identity can be, for example, at least about 50%, 55%, 60%, 65%, 70%, 75%, 80%, 85%, 90% 91%, 92%, 93%, 94%, 95%, 96%, 97%, 98%, 99%, or 100% amino acid identity to the given polypeptide, oligopeptide, peptide or protein. Also included within the definition are polypeptides, oligopeptides, peptides and proteins that have one or more conservative amino acid substitutions as compared to a given polypeptide, oligopeptide, peptide or protein. It is understood that the polypeptides can occur as single chains or associated chains. Methods for making polypeptides, oligopeptides, peptides and proteins are known in the art.

The term “bind”, in the context of, for example, a target binding domain (TBD) comprising at least one moiety that binds to a target protein, means an amino acid residue of the TBD that participates in an electrostatic interaction with the target protein, participates in a hydrogen bond with the target protein, or participates in a water-mediated hydrogen bond with the target protein, or participates in a salt bridge with the target protein, or it has a non-zero change in buried surface area due to interaction with the target protein, and/or a heavy atom of the TBD is located within 4 Å of a heavy atom of a residue of the target protein.

The following symbol:

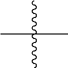

is used in chemical structures herein to represent a point of covalent attachment of a group to another group.

### General Techniques

The practice of the present disclosure will employ, unless otherwise indicated, conventional techniques of synthetic chemistry, organic chemistry, biochemistry, molecular biology, cell and tissue culture, immunology, genetics, etc. which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999); and Immunobiology (C. A. Janeway and P. Travers, 1997).

### Multivalent Antibody Recruitment Molecules

The present disclosure provides multivalent ARM compounds comprising a plurality of TBDs that bind to two or more different target proteins expressed by a cancer, at least one ABD and/or cytotoxic agent, and a polymer scaffold connecting the plurality of TBDs with the at least one ABD and/or cytotoxic agent.

The plurality of TBDs comprises ligands that bind to two or more different target proteins such as, but not limited to, PSMA, uPAR, HER2, EGFR, CD38, BCMA and/or integrins.

In certain embodiments, the TBDs comprise:

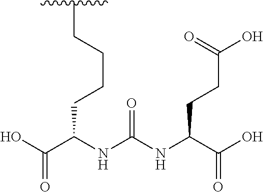 [SMILES: *CCCC[C@H](NC(=O)N[C@@H](CCC(=O)O)C(=O)O)C(=O)O]

to target PSMA;

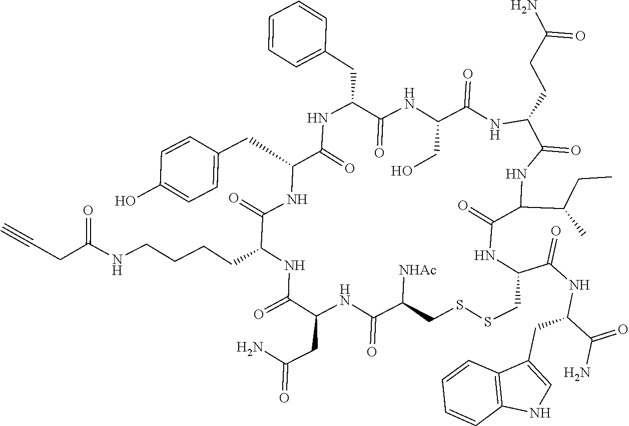 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]

or azido-K-G-S-G-G-D-Cha-F-s-r-Y-L-W-S to target uPAR, wherein Cha is cyclohexylalanine, L-amino acids are shown in upper case and D-amino acids are shown in lower case; and/or

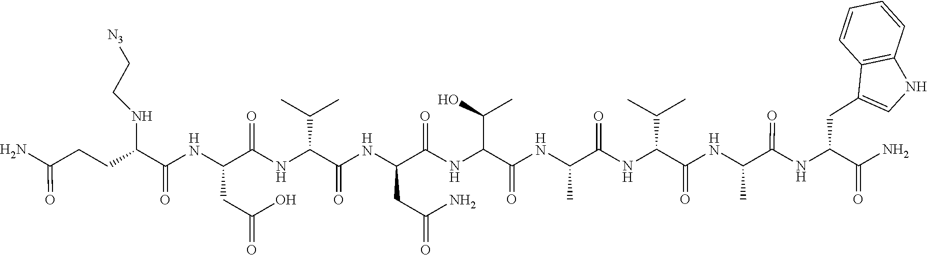 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

to target HER2.

The ABD is, for example, a pan IgG binding ligand. In certain embodiments, the ABD comprises:

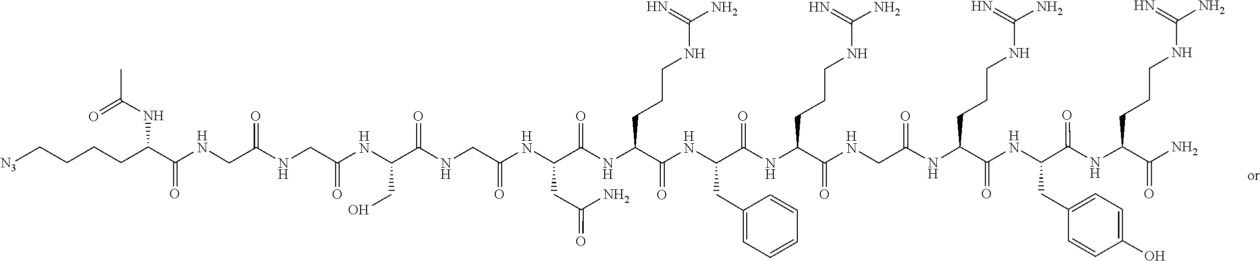 [SMILES: CC(=O)N[C@@H](CCCCN=[N+]=[N-])C(=O)NCC(=O)NCC(=O)N[C@@H](CO)C(=O)NCC(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](Cc1ccc(O)cc1)C(=O)N[C@@H](CCCNC(=N)N)C(N)=O]

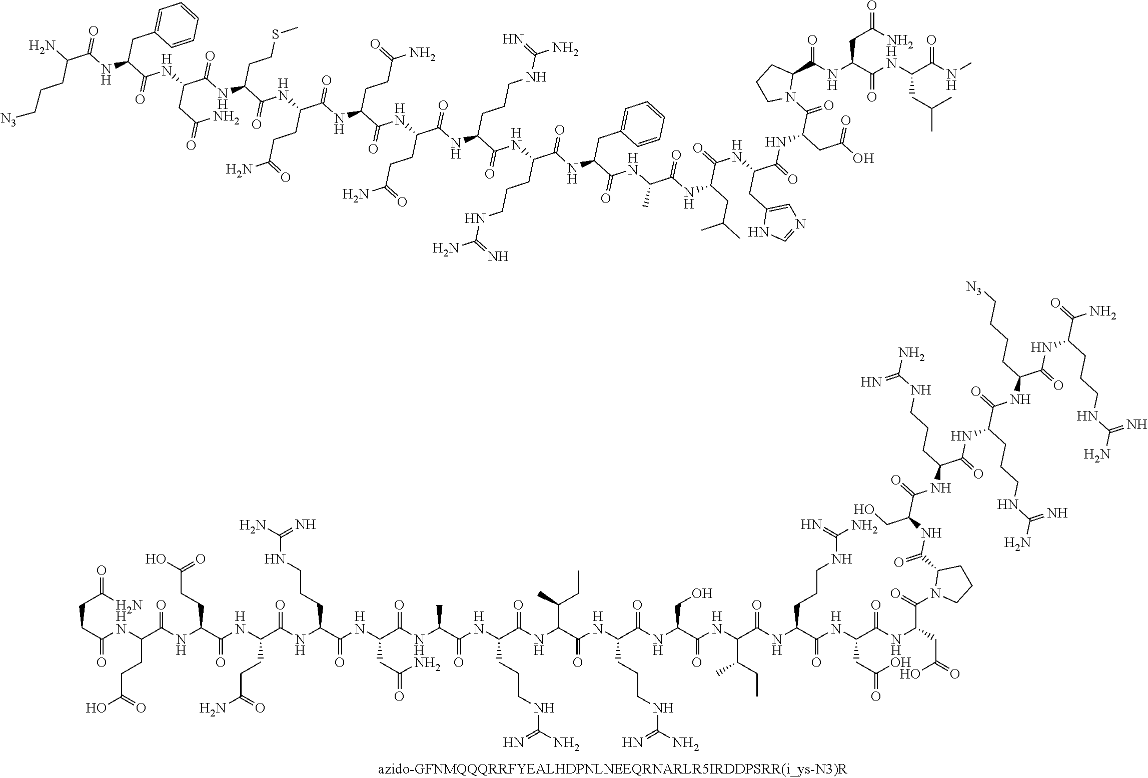

In an embodiment, the multivalent antibody recruitment molecule is:

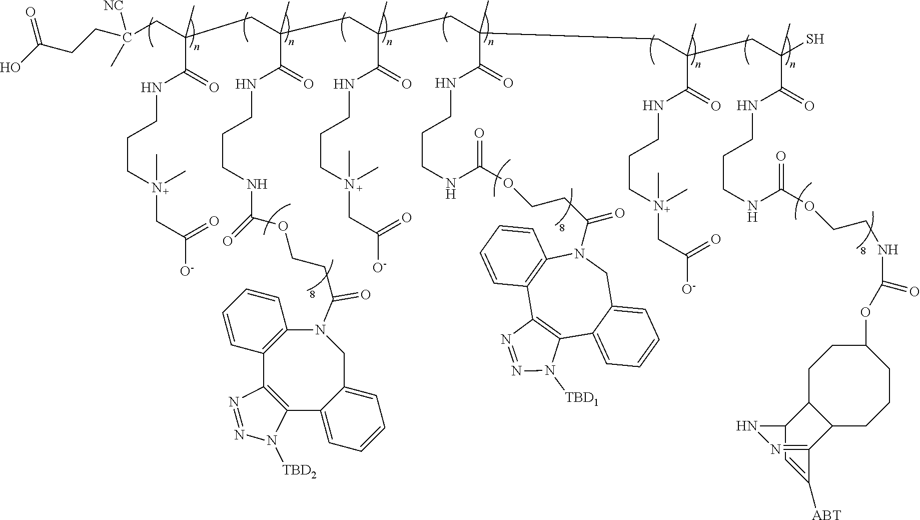

wherein n is an integer between 1 and 50,
or a pharmaceutically acceptable salt or solvate thereof.

### Methods of Preparing Multivalent Antibody Recruitment Molecules

The multivalent ARM compounds disclosed herein can be prepared by various synthetic processes. The choice of particular structural features and/or substituents may influence the selection of one process over another. The selection of a particular process to prepare an ARM compound is within the purview of the person of skill in the art. Some starting materials for preparing compounds of the present disclosure are available from commercial chemical sources. Other starting materials, for example as described below, are readily prepared from available precursors using straightforward transformations that are well known in the art.

The compounds of the invention may be synthesized according to the methods and protocols disclosed herein. In an embodiment, the compounds may be synthesized using standard chemical connectivity between the polymer scaffold, the TBD(s) and the ABD(s), along with appropriate protecting groups when necessary. In an embodiment, the approach uses standard functional group chemistry.

In an embodiment, the ABD comprises a peptide which may be synthesized using, for example, solid phase peptide synthesis (SPPS). In an embodiment, the ABD comprises a peptide which may be expressed/overexpressed in cells using standard molecular biology techniques, and then purified using standard peptide purification protocols known in the art.

Standard functional group chemistries, that may be used in the preparation of compounds of the application, include, for example, coupling a carboxylic acid to either an amine or an alcohol to generate esters or amides through standard carbodiimide conditions (e.g., DCC, EDCI, DIC) along with base and catalytic amine (e.g., DMAP, imidazole), or by conversion to the acid chloride through oxalyl chloride or thionyl chloride, etc., followed by addition of amine/alcohol.

Additionally, for example, an amine or an alcohol may be coupled to an isocyanate or an isothiocyanate to generate ureas, thioureas, or the corresponding carbonates or thiocarbonates.

Still, in a further approach, for example, a heterolinker can be made through treating a nucleophile with the appropriate leaving group. Some leaving groups could be halogens, such as bromine, or sulfonates, such as triflates or tosylates.

The multivalent ARM compounds of the disclosure may include one or more pharmaceutically acceptable salts. The formation of a desired compound salt is achieved using standard techniques. For example, the neutral compound is treated with an acid or base in a suitable solvent and the formed salt is isolated by filtration, extraction or any other suitable method. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxyalkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N, 1\r-dibenzylethylenediamine, N-methylglucamine, chloroprocaine, choline, diethanolamine, ethylenediamine, procaine and the like.

The multivalent ARM compounds of the disclosure may be solvates of the multivalent ARM compounds. The term “solvate” as used herein means a compound, or a salt of a compound, wherein molecules of a suitable solvent are incorporated in the crystal lattice. The formation of solvates of the compounds provided herein will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

As used herein “pharmaceutically acceptable” means compatible with the treatment, diagnosis or analysis of subjects.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations-A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by those skilled in the art.

### Compositions Comprising Antibody Recruitment Molecules

The multivalent ARM compounds of the present invention may be in the form of a pharmaceutical composition comprising the multivalent ARM compound and at least one pharmaceutically acceptable carrier, diluent, excipient or stabilizer (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable carriers, diluents, excipients and stabilizer are further described herein.

The multivalent ARM compounds and compositions thereof can also be used in conjunction with other agents that serve to enhance and/or complement the effectiveness of the agents.

A pharmaceutical composition of the disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and non-aqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be suitable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Without being bound by any theory, the ARMs of the present invention that have at least two or more different TBDs allow for improved immune-mediated clearance of a cancer cell expressing a protein targeted by the TBD. As cancer cells change the antigens expresses on their surface, they develop resistance to various therapies. The ARMs of the present invention target more than one antigen, so even if the cancer cell stops expressing one antigen (e.g., an antigen that binds TBD1), the ARM can still bind a different antigen (e.g., via TBD2) and so the immune-mediated clearance of the cancer cell continues. In addition, having more than one copy of each TBD on the ARM increases the binding avidity of the ARM, allowing for enhanced immune elimination of tumors that express antigens at lower levels, compared to ARMs that contain only a single TBD.

### EXAMPLES

The disclosure is further described by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Example 1: Targeting Lower Antigen Expressing Cancers with Multivalent ARMs

Chemical immunotherapy has provided the means to direct endogenous immune machinery to chosen disease targets for immune-mediated target clearance without the need for costly and arduous cell and/or protein engineering. One such chemical immunotherapeutic is a class of bivalent small molecules (about 1 kDa) known as Antibody Recruiting Molecules (ARMs). ARMs contain an antibody-binding domain (ABD) and a target-binding domain (TBD) to direct endogenous serum antibodies (Abs) to highly overexpressed cancer targets for immune mediated target clearance. ARM function relies on a high valency of cancer targets on the cell surface which poses an issue for tumors displaying a lower valency of the target or undergoing single antigen down-regulation. To address this limitation and amplify antibody recruitment to lower valency cancer targets, a tunable multivalent immune recruitment platform (MIR) was developed. In this platform, polymeric ARMs (pARMs) were assembled that make multivalent potential high avidity binding contacts with both serum antibodies and cancer targets. PARMs, generated from monomeric units using RAFT chemistry, were designed to recruit multiple anti-dinitrophenol (DNP) antibodies to multiple prostate specific membrane antigens on prostate cancer cell lines. The tunablility of this platform was demonstrated by efficiently screening effects of ABD: TBD ratio, ligand flexibility, and ligand copy on anti-cancer immune function. Optimized PARMs were observed to mediate enhanced anti-cancer immune function compared to analogous monovalent ARMs, when targeting lower antigen expressing prostate cells. The MIR platform may be used in mechanistic studies interrogating avidity effects underlying immune activation, and as a therapeutic strategy that uniquely exploits disease cell receptor density to affect selective proximity induction with immune cells.

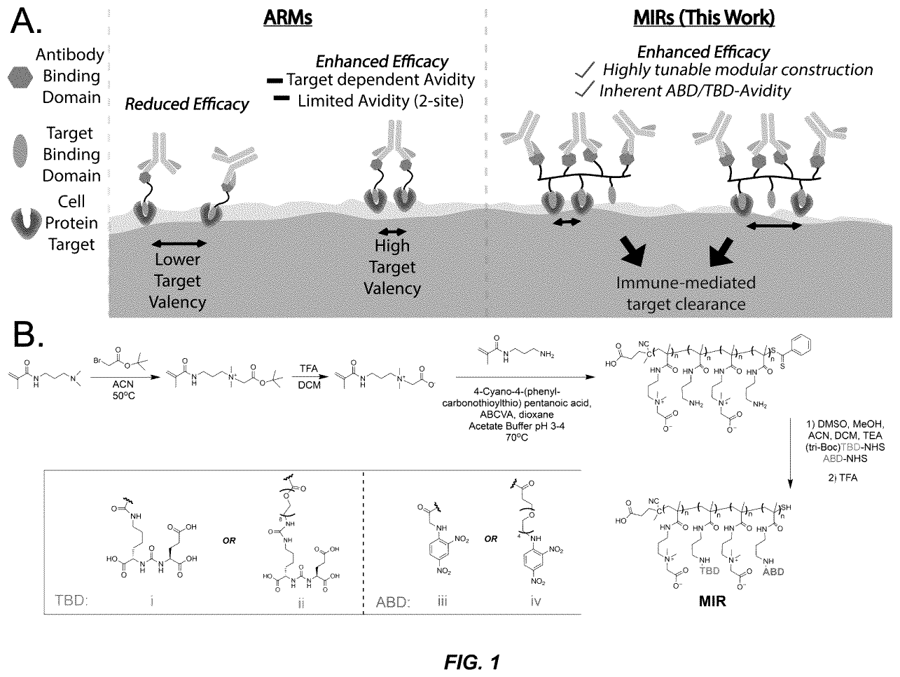

ARM function relies on high avidity binding to both recruited antibodies and tumor antigens, which is promoted on cancers that express high densities of cell surface tumor antigen (approx. 105-106 copies/cell), (FIG. 1A). As tumor antigen density decreases, ARMs lose apparent affinity for both antibody and the tumor cell as antibody facilitated bi-valent avidity binding (two separate ARM binding events antibody Fabs and two closely packed targets) is disfavored. Most cancers, however, are associated with variable and often lower tumor antigen expression which can limit ARM function. Low affinity anti-ABD antibodies endogenous to human serum (kd˜10⁻⁵-10⁻⁷M) coupled with fast clearance of ARM from systemic circulation may further limit their efficacy.

To enhance binding affinity for serum antibodies and their recruitment to the tumor, recent macromolecule-based multivalent-ABD strategies have been developed in the context of intra-tumor injection (Uvyn et al, “Cell surface clicking of antibody-recruiting polymers to metabolically azide-labeled cancer cells” (2019) Chem Commun (Camb) 55 (73): 10952-10955). Here the macromolecule contains multiple ABD binding units to increase antibody binding affinity via avidity. Since the therapeutic is injected directly into the tumor, TBD ligands are not required. In a scenario where systemic therapeutic delivery is desirable however, a multivalent antibody recruiting macromolecule requires TBT units. To target lower antigen expressing tumor cells with enhanced efficacy compared to monovalent ARMs, a tunable multivalent immune recruitment (MIR) platform was developed that uses polymers to present optimal arrays of TBD and ABD ligands. The resultant polymeric ARMs (PARMs) can be tuned to engage both serum antibodies and lower antigen expressing tumor cell with higher avidity, compared to monovalent ARMs. This is because pARMs can contact tumor antigens spaced farther apart on tumor cells with a lower antigen density compared to ARMs (FIG. 1A). Additionally, in contrast to ARMs, avidity binding to antibody is no longer dependent on the number of tumor antigens and is fixed by the pARM ABD spacing. High avidity pARM binding to cancer cells can lower therapeutic dosing, and enhance target residence time to promote efficient immune cell engagement. Notably, avidity binding to high tumor antigen expressing cells has been reported previously by Csizmar et al, “Multivalent Ligand Binding to Cell Membrane Antigens: Defining the Interplay of Affinity, Valency, and Expression Density” (2019) J Am Chem Soc 141 (1): 251-261, using TBD functionalized macromolecular protein “nanorings”.

PARMs were synthesized using a single linear synthetic polymer (LSP) backbone (FIG. 1B). The use of a LSP provides a large scaffold to limit rapid serum clearance as well as increased conformational flexibility/diversity, synthetic scalability, and resistance to reticular endothelial clearance compared to spherical scaffolds such as liposomes/nanoparticles. Further, LSPs remain resistant to enzymatic degradation possible with biopolymers. Finally, the ability of LSPs to present a large linear array of binding ligands is theoretically predicted to enable optimal discrimination of cell surfaces that differ in target receptor densities, a principle known as super-selectivity. This is particularly strategic for multivalent immune recruiting to lower antigen expressing tumor cells, where normal healthy cells express the same antigen at trace levels. PARMs were synthesized to contain glutamate-urea-lysine (GUL) TBDs for targeting prostate specific membrane antigen (PSMA) on prostate cancer and general tumor neovasculature. As an ABD, the established dinitrophenol (DNP) ligand was used. The MIR pARM backbone was synthesized as a co-polymer of carboxybetaine (providing zwitterionic/non-fouling/stealth character to the polymer) and aminopropyl (to allow for specific modification with ligands of choice). Through optimization of ABD/TBD number/ratio and flexibility, PARMs may target tumor cells expressing lower antigen expression levels (i.e., ≤105/cell) with increased avidity leading to enhanced immune elimination, compared to monomeric ARMs.

The effects of ligand copy number, ABD: TBD ratio, and flexibility of ligand presentation on pARM immunotherapeutic function were examined (FIG. 1). The backbone polymer was synthesized using RAFT chemistry to yield a 35 kDa unmodified polymer with PDI 1.3 (Appendix). Differing ratios of DNP and GUL derivatives were appended onto the polymer backbone simultaneously via NHS chemistry using strict stoichiometric control (Table 1 below). A peg extender between polymer and ligand was included or omitted to study ligand flexibility influence on binding and function. Subsequent molecular weight of each MIR is over the proposed renal filtration threshold of approximately 40 kDa. As a monomeric comparison an ARM with equivalent ABD/TBD to each MIR was included in all analysis (Appendix Scheme S1).

TABLE 1
Composition of Constructs

| | | | | | # | | # |
|---|---|---|---|---|---|---|---|
| | MW | | | % | TBD | % | ABD |
| Construct | (kDa) | TBD | ABD | TBD^{b} | per^{ab} | ABD^{b} | per^{ab} |
| M40 | 49 | i | iii | 66% | 30 | 34% | 15 |
| MP40 | 57 | ii | iii | 60% | 24 | 40% | 16 |
| MP80 | 52 | ii | iii | 21% | 10 | 79% | 40 |
| MPP40 | 63 | ii | iv | 66% | 29 | 34% | 15 |
| ARM | 1 | ii | iii | N/A | 1 | N/A | 1 |

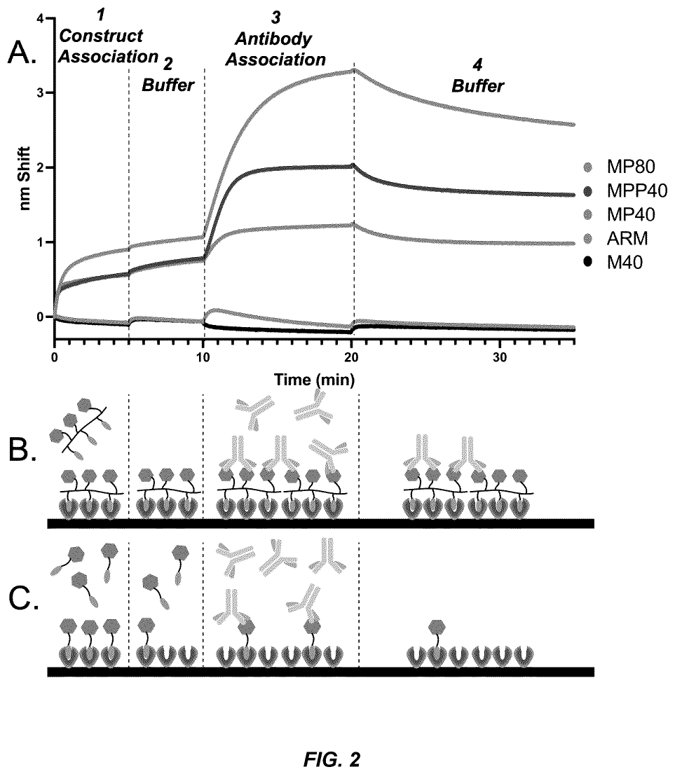

To evaluate each MIR's capacity to bind PSMA with avidity and recruit anti-DNP antibody to a target surface, biolayer interferometry (BLI) was used (FIG. 2). Probes coated with immobilized PSMA were dipped into 1 uM solution of each construct during an initial stage of “Construct Association” (FIG. 2). M40 failed to bind the probe due most likely to polymer steric bulk proximal to TBD units. This result demonstrates the necessity of a flexible peg spacer between TBD and polymer backbone for MIR-PSMA binding. ARM did not induce binding signal due to low molecular weight but was bound to the probe (binding confirmed in subsequent steps).

In the subsequent “Buffer” step (FIG. 2), probes were submerged in a wash solution to dissociate construct-PSMA binding not enhanced through avidity. The lack of dissociation of all associated MIR constructs suggests rapid microscopic ligand-PSMA re-binding events before macroscopic construct dissociation (i.e., PSMA binding is enhanced by avidity). Construct coated probes were then submerged into a 50 nM anti-DNP antibody solution to measure capacity of retained construct to recruit antibody. MP80 demonstrated the most antibody recruitment followed by MPP40, in line with DNP incorporation. MP40 demonstrated abrogated antibody recruiting potential compared to MPP40. This indicates extension of DNP from parent backbone significantly aids antibody recruitment in the MIR system. Abrogation of antibody binding to MP80 did not appear significant. Without being limited to any theory, the increased DNP on MP80 may compensate for steric antibody occlusion and enhance antibody recruitment via avidity. Abrogated antibody recruitment by ARMs was attributed to ARM dissociation from PSMA in the prior “Buffer” step, coupled with dissociation of both ARM and/or ARM-antibody complex during the antibody association phase.

Probes were washed again to dissociate construct-antibody complexes lacking stabilization via avidity. Each of MP80, MPP40, and MP40 display a loss of binding amplitude of approx. 30%, 30%, 50% respectively, followed by a plateau. This initial dissociation phase was attributed to monomeric binding events between antibody and MIR. Extracting K_{off} of each dissociation phase allows estimation of monomeric (non-avidity enhanced) antibody-MIR K_{d}, which are all in close agreement with the K_{d} of this antibody for monomeric DNP (on the order of K_{d}˜ 108 M), (Appendix Figure S2). Premature leveling of dissociation plateau is consistent with strong avidity binding of antibody-MIRs. Due to the very small k_{off} in this plateau region, only an upper limit can be defined for apparent avidity enhanced binding affinity K_{d}^{avid}<10⁻⁹ M. Notably, MP80 not only recruited more antibody overall but also bound more antibody with avidity stabilization. This may enable for amplification of antibody recruitment to lower antigen expressing prostate cancer cells compared to ARMs. MP80 however is most at risk for off-target immune activation.

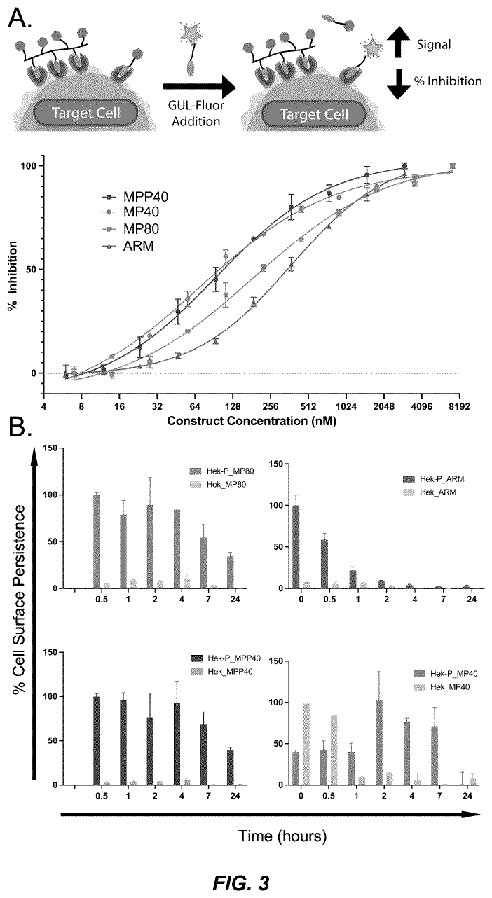

Unlike immobilized PSMA on BLI probes, PSMA anchored on a cancer cell membrane is fluid and dynamic. As such, flow cytometry assays were developed to evaluate MIR binding to PSMA directly on a model cancer cell line engineered to express a high valency of PSMA (>105 PSMA/cell, Hek-P) (FIG. 3). The ability of MIRs or ARM to complete with a fluorophore labeled ARM “tool” compound for binding to cell surface PSMA was measured. (FIG. 2A, Scheme S1). Each MIR was incubated with target cells at increasing concentrations for 20 minutes at 4° C. to inhibit endocytosis. GUL-Fluor (400 nM) was added directly, and competitive binding was monitored via flow cytometry (FIG. 3A). MIRs displaced GUL-fluor and bound to PSMA with significantly higher affinity compared to monomeric ARM. Further, MIRs with higher TBD substitution (MP40, MPP40) inhibited GUL-Fluor more effectively than MP80. These findings are consistent with MIRs binding simultaneously to multiple PSMA receptors resulting in high avidity binding to the cell. Applying the Cheung-Prusoff equation using calculated IC₅₀ values yields Kᵢ of each construct in the presence of monomeric competitor (Appendix Table S2). Here MP40 and MPP40 demonstrated ˜4-5-fold increase to Kᵢ (for PSMA binding) compared to the monomeric ARM. Notably, MIR binding avidity is likely underestimated in this assay since the competitor molecule GUL-fluor inhibits TBD rapid re-binding events. This effect can differentially accelerate MIR dissociation from the cell relative to ARMs.

Multivalent high avidity binding of MIRs to cancer cells should decrease cell-surface dissociation kinetics (increase residence time). To evaluate MIR vs ARM residence time, dissociation of recruited antibody to model Hek-P cells was measured as a function of time (FIG. 3B). To promote dissociation and potentially model in vivo clearance of free MIR/ARM, buffer wash steps were included in the assay. Notably, in vivo clearance will drive MIR/ARM dissociation from the cell decreasing therapeutic efficacy. Briefly, target cells were incubated with each construct (200 nM) at 4° C. for 20 minutes then washed to remove unbound construct. Cells were then incubated for up to 24 hours in complete media to allow construct dissociation. Construct residency was probed via a primary anti-DNP antibody (50 nM)/fluorescent secondary antibody pair on flow cytometry. Specificity is demonstrated with an isogenic control PSMA (-) cell line (Hek). Monomeric binding ARM showed almost complete depletion from cell surface within 1 hour. Both MP80 and MPP40 demonstrated ˜20× enhancement over monomeric ARM, showing significant persistence on the cell surface for up to 24 hours. In line with previous experiments, these results support avidity stabilized MIR binding to cell surface PSMA with enhanced affinity compared to ARMs. MP40 demonstrated non-specific target binding within the first hour. In line with previous results, MP40 function may be impaired due to sub-optimal ligand presentation that is restored by incorporation of the PEG spacer in MPP40.

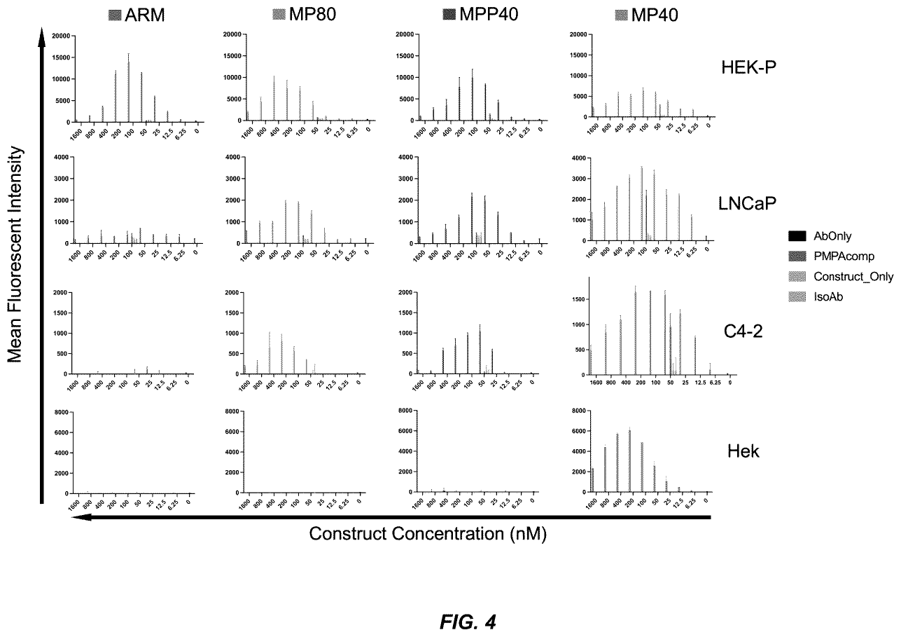

Antibody recruitment by MIRs and ARMs to therapeutically relevant lower antigen expressing prostate cancer cells on the order of ˜ 104-105 receptors/cell was evaluated. MIRs may bind both tumor cells and serum antibody with higher affinity due to avidity as described above (FIG. 1A). Each construct was incubated in a dilution series with anti-DNP antibody (50 nM) and target cells expressing high (Hek-P), moderate (LNCaP/C4-2), or no PSMA (Hek) (Appendix Figure S4). Antibody recruitment was quantified via addition of fluorescent secondary antibody and measurement by flow cytometry (FIG. 4). In line with previous results, all constructs demonstrated high capacity to recruit antibody to high PSMA expressing (HEK-P) cell line, with MP40 demonstrating slightly attenuated function. On moderate PSMA expressing cell lines (LNCaP and C4-2), however, ARM mediated antibody recruiting was significantly abrogated compared to each MIR. Each MIR demonstrated significant capacity to recruit antibody to these moderate PSMA expressing cells even at low nM concentrations. Avidity-based target binding along with avidity-based antibody binding intrinsic to MIRs allows for measurable antibody recruitment with fewer target antigens available, and fewer constructs on target surface. Recruitment to isogenic PSMA (-) Hek cell line demonstrates specificity of both MP80 and MPP40. However, in line with previous results, MP40 antibody recruitment was largely non-specific, particularly at high concentrations. The concentration dependence on non-specific binding might suggest instability of MP40 is driven by intermolecular aggregation alleviated by a flexible linker acting as an entropic hurdle. In all cases, controls included PMPA competition (PMPAcomp; competition for specific PSMA binding) and non-DNP binding isotype IgG antibody (IsoAb). These controls comment on construct specificity to both target and antibody, further demonstrating MP40 target binding was largely non-specific.

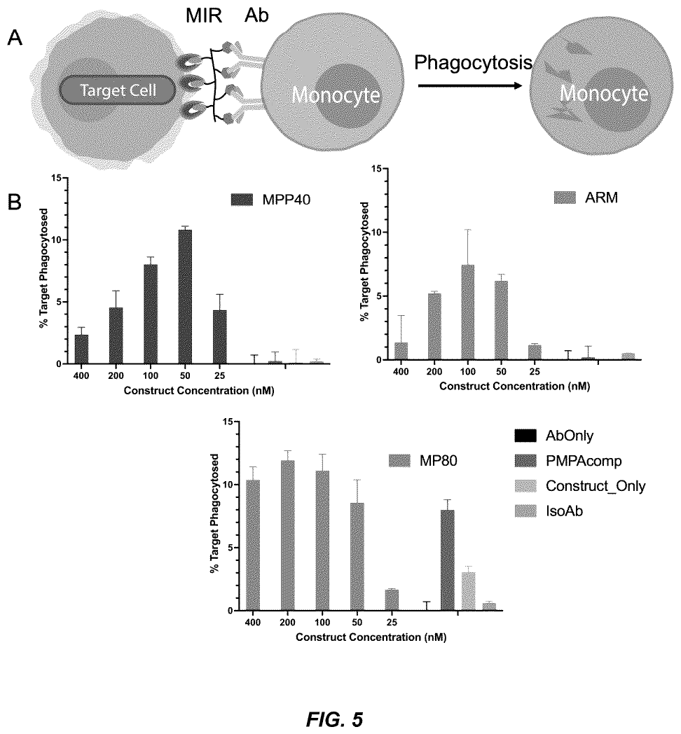

Finally, the ability of each MIR to induce immune-mediated cell clearance via antibody dependent cellular phagocytosis (ADCP) was investigated (FIG. 5). In these experiments, MP40 was excluded due to non-specificity. A constant physiologically relevant antibody concentration (50 nM) with a construct dilution series was applied to DiO stained cell line C4-2. DiD stained U937 monocytes were immediately added to this mix followed by a 1-hour incubation at 37° C. ADCP was measured via flow cytometry (Appendix Figure S5). MP80 induced ADCP of target cell line in the presence of PSMA competition (PMPAcomp) and with PSMA (-) Hek cells, (Appendix Figure S6). This observation supports MP80 induces antibody dependent clustering of Fc receptors leading to target independent immune activation. This phenomenon has not been explored or reported in the literature for other macromolecular antibody recruiting therapeutic systems. Analogous control experiments using both ARM and MPP40 however demonstrated specific ADCP (FIG. 5; Appendix Figure S6). Without being limited to any theory, the ability of ARM to illicit ADCP with no observable antibody recruiting (FIG. 4) may be due to the reduced binding threshold for inducing ADCP in in vitro assays vs detection of antibody recruitment in flow cytometry. The ADCP assay may also amplify signal through multiple cycles of phagocytosis per construct, whereas antibody recruitment has only one signal (fluorophore) per construct. In these experiments, MPP40 demonstrated a significant advantage over monovalent ARMs at mid to low construct concentrations (<100 nM). Enhancement of specific immune mediated clearance is consistent with increased K_{dapp} for target PSMA and antibody via avidity driven multivalent binding.

A new tunable MIR platform is provided for selective amplification of antibody recruitment to lower antigen expressing tumor cells, compared to monovalent ARMs. This amplification arises due to avidity enhanced binding to both PSMA on prostate cancer cells and anti-DNP antibody. Modular co-polymer MIR construction coupled with amine-NHS conjugation chemistry allowed for efficient construct optimization and subsequent functional analysis. Such analysis revealed the critical importance of flexible extenders between MIR backbone and both ABD/TBD ligands for optimal targeting and specificity. Additionally, off target MIR-induced immune activation was demonstrated when valency for antibody recruitment is too high.

Further supporting data and detailed experimental methods for Example 1 are included in the Appendix. The Appendix forms part of the present disclosure.

### Example 2: Synthesis of Multivalent ARMs

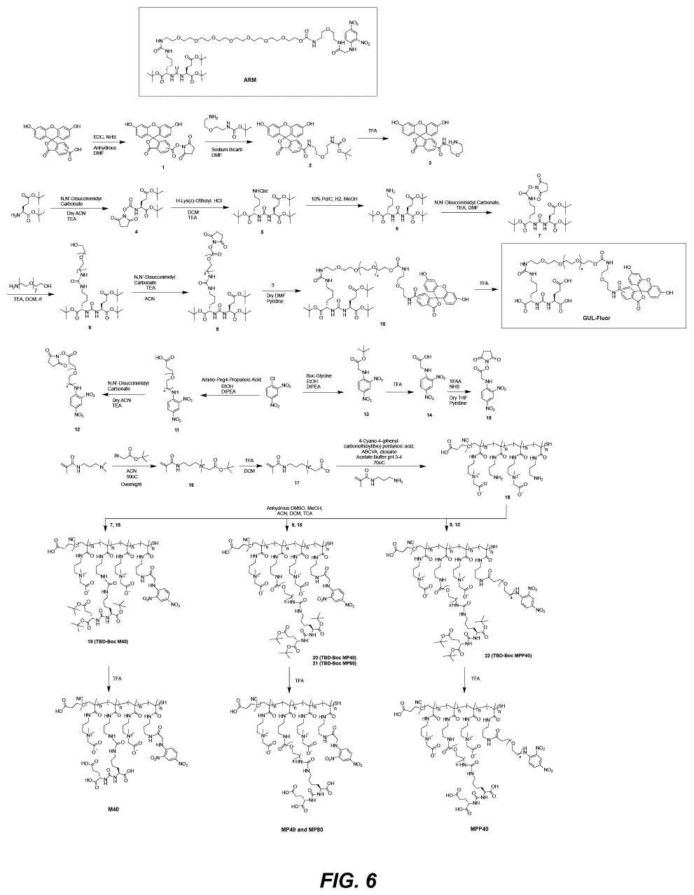

Multivalent ARMs of the present invention are synthesized essentially as shown in FIG. 6 (and described in further detail in the Appendix, including Scheme S1 and S2), which shows synthesis of an ARM comprising a plurality of TBDs that bind to PSMA and ABDs that bind to anti-DNP antibodies using methods known to those skilled in the art.

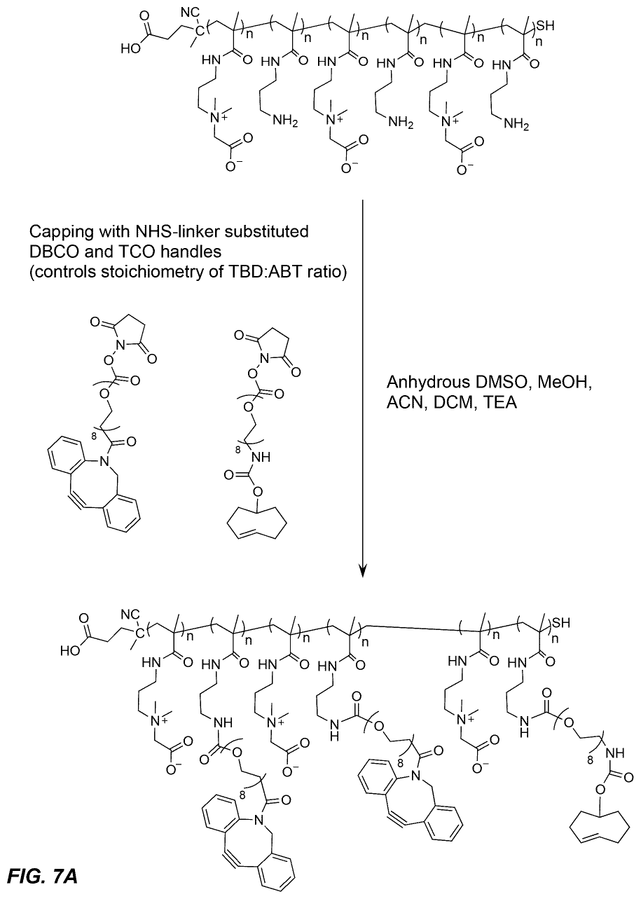

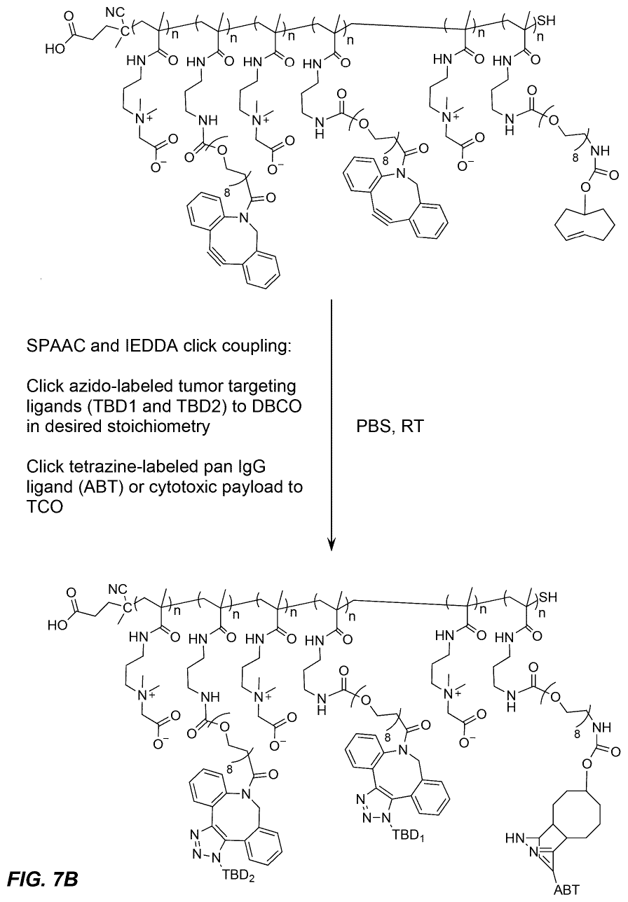

Multivalent ARMS of the present invention having two or more different TBDs (e.g., TBD1 and TBD2) are synthesized essentially as shown in FIGS. 7A and 7B, using methods known to those skilled in the art. Each TBD is prepared as an azide functionalized conjugate of small molecule or peptide ligand (e.g., a uPAR binding peptide such as azido-K-G-S-G-G-D-Cha-F-s-r-Y-L-W-S to target uPAR, wherein Cha is cyclohexylalanine, L-amino acids are shown in upper case and D-amino acids are shown in lower case; or a HER2 binding peptide such as:

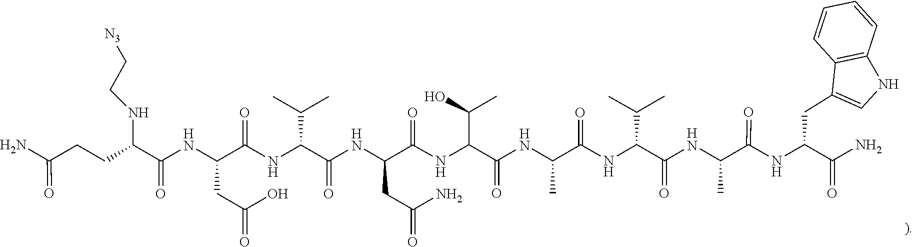 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

In this synthesis, the number and ratio of TBD1: TBD2 is tuned by changing reactant stochiometry during the strain-promoted azide-alkyne click (SPAAC) reaction.

The pan IgG ligand

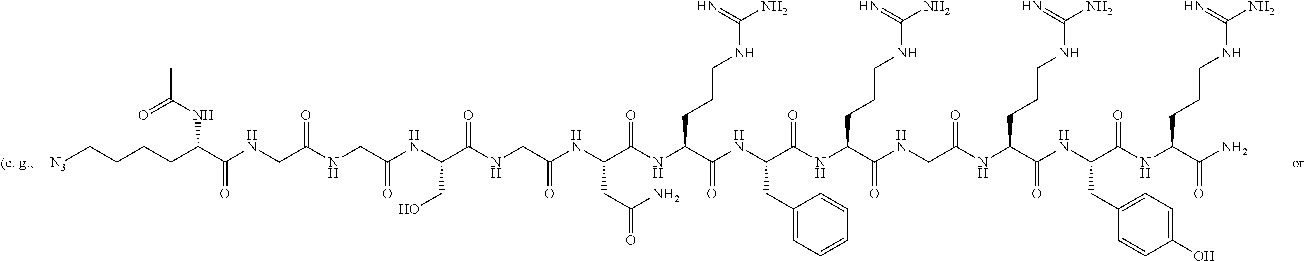 [SMILES: CC(=O)N[C@@H](CCCCN=[N+]=[N-])C(=O)NCC(=O)NCC(=O)N[C@@H](CO)C(=O)NCC(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](Cc1ccc(O)cc1)C(=O)N[C@@H](CCCNC(=N)N)C(N)=O]

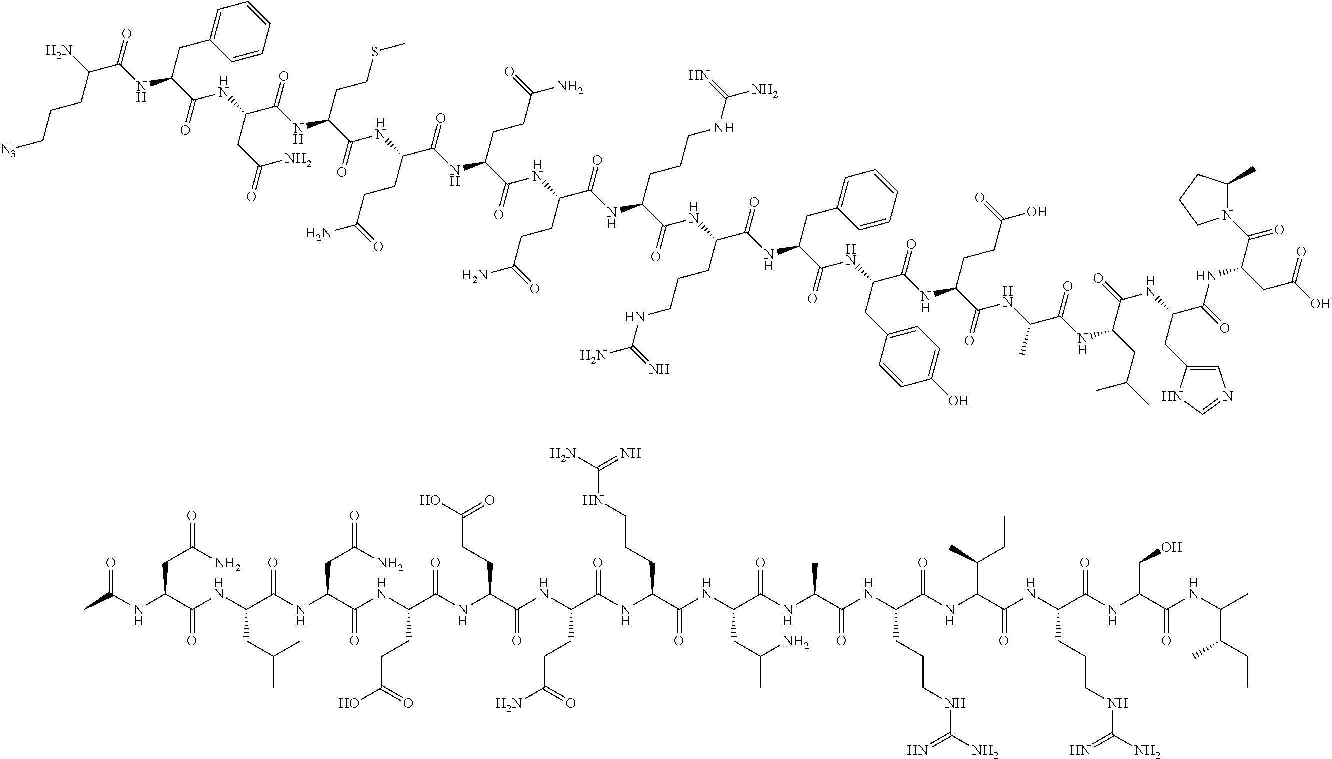

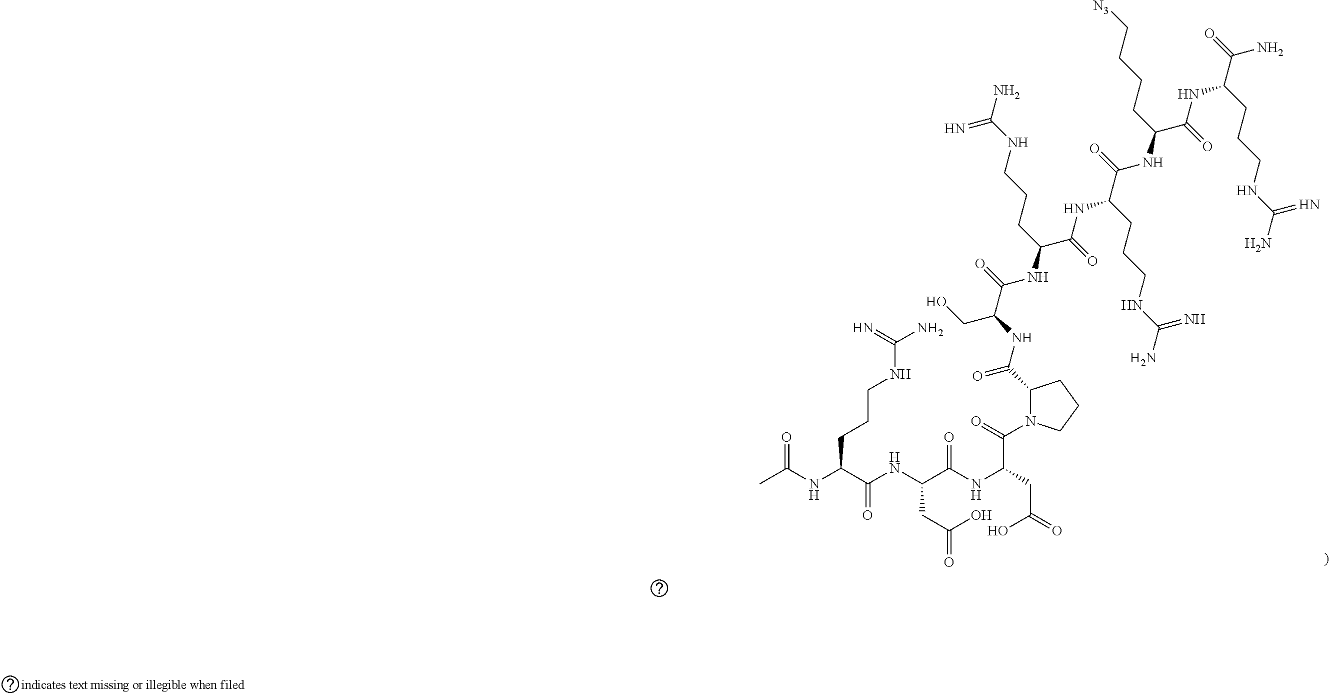 [SMILES: CC(=O)N[C@@H](CCCNC(=N)N)C(=O)N[C@@H](CC(=O)O)C(=O)N[C@@H](CC(=O)O)C(=O)N1CCC[C@H]1C(=O)N[C@@H](CO)C(=O)N[C@@H](CCCNC(=N)N)C(=O)N[C@@H](CCCNC(=N)N)C(=O)N[C@@H](CCCCN=[N+]=[N-])C(=O)N[C@@H](CCCNC(=N)N)C(N)=O]

or a cytotoxic payload is orthogonally installed via an inverse electron demand Diels-Alder (IEDDA) click chemistry.

Although the disclosure has been described with reference to certain specific embodiments, various modifications thereof will be apparent to those of ordinary skill in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all documents cited herein are incorporated herein by reference as if set forth in their entirety.

We claim:

1. A compound comprising:
a plurality of target binding domains (TBDs) that bind to two or more different target proteins expressed by a cancer;
at least one antibody binding domain (ABD) and/or cytotoxic agent; and
a polymer scaffold connecting the plurality of TBDs with the at least one ABD and/or cytotoxic agent.

2. The compound of claim 1, wherein the target proteins comprise two or more of PSMA, uPAR, HER2, EGFR, CD38, BCMA and integrins.

3. The compound of claim 1, wherein the TBDs comprise: 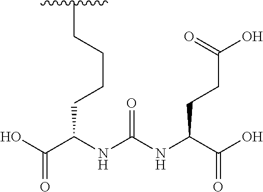 [SMILES: *CCCC[C@H](NC(=O)N[C@@H](CCC(=O)O)C(=O)O)C(=O)O]
to target PSMA; 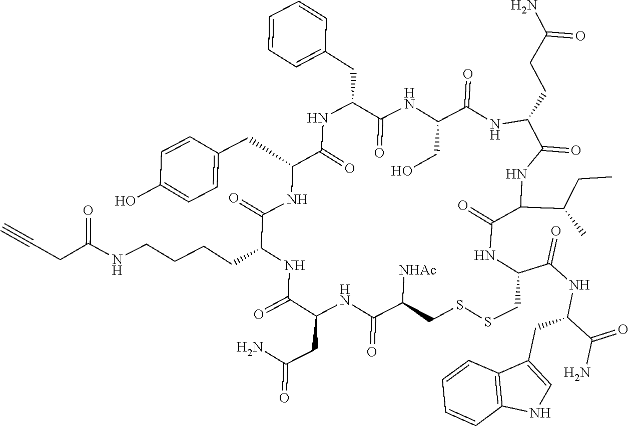 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]
or azido-K-G-S-G-G-D-Cha-F-s-r-Y-L-W-S to target uPAR, wherein Cha is cyclohexylalanine, L-amino acids are shown in upper case and D-amino acids are shown in lower case; and/or 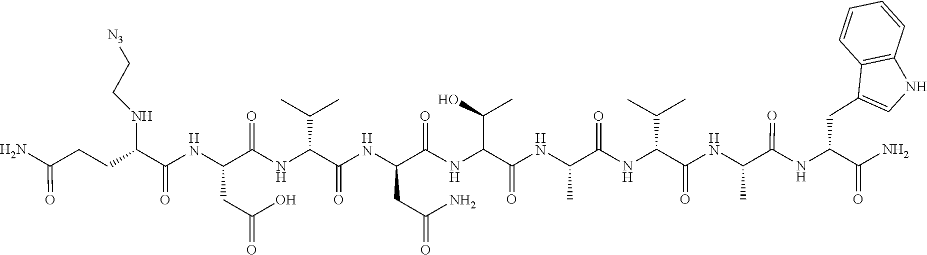 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]
to target HER2.

4. The compound of claim 1, wherein the cancer is breast cancer, multiple myeloma, prostate cancer, or glioblastoma.

5. The compound of claim 1, wherein:
the cancer is prostate cancer and the target proteins comprise PSMA and HER2;
the cancer is prostate cancer and the target proteins comprise PSMA, HER2 and uPAR;
the cancer is glioblastoma and the target proteins comprise two or more of HER2, uPAR, PSMA and EGFR;
the cancer is multiple myeloma and the target proteins comprise CD38 and BCMA; or
the cancer is breast cancer and the target proteins comprise two or more of HER2, uPAR and EGFR.

6. The compound of claim 1, wherein the cancer is a HER2-positive breast cancer and/or wherein the cancer is a triple negative breast cancer.

7. The compound of claim 1, wherein the ABD is a pan IgG binding ligand and the pan IgG binding ligand recruits serum IgG to the cancer.

8. The compound of claim 7, wherein the pan IgG binding ligand is

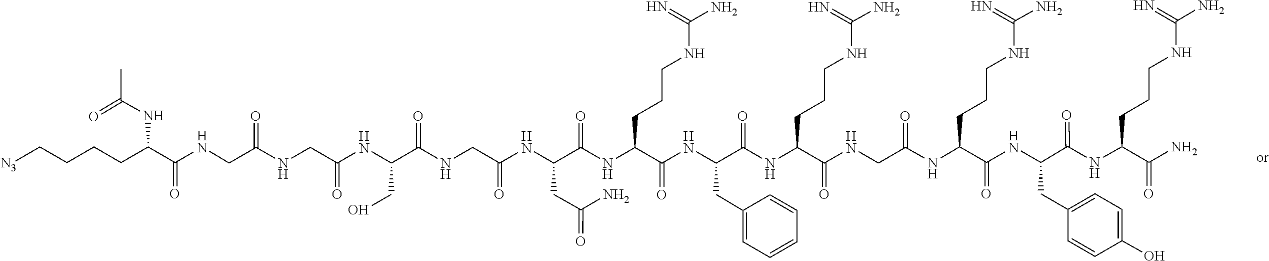 [SMILES: CC(=O)N[C@@H](CCCCN=[N+]=[N-])C(=O)NCC(=O)NCC(=O)N[C@@H](CO)C(=O)NCC(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](Cc1ccc(O)cc1)C(=O)N[C@@H](CCCNC(=N)N)C(N)=O]

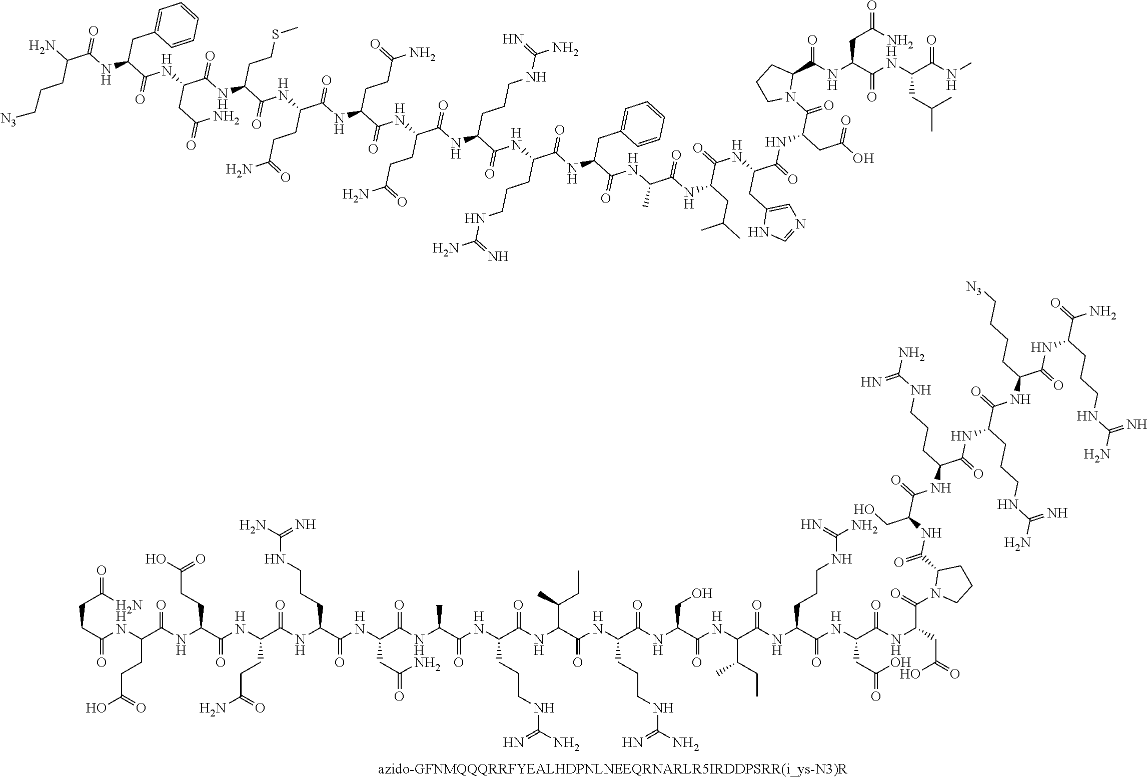

9. The compound of claim 1, wherein the cytotoxic agent is doxorubicin, a topoisomerase inhibitor, or a mitotic inhibitor.

10. The compound of claim 1, wherein the polymer scaffold comprises a ROMP block co-polymer, a RAFT PCB/methyl acrylate co-polymer or a synthetic nucleic acid biopolymer.

11. The compound of claim 1, wherein the ratio of TBDs: ABDs is between 1:10 and 10:1.

12. The compound of claim 1, wherein the plurality of TBDs comprises a first TBD (TBD1) and a second TBD (TBD2) and wherein the ratio of TBD1: TBD2 is between 1:10 and 10:1.

13. The compound of claim 12, wherein the compound is: 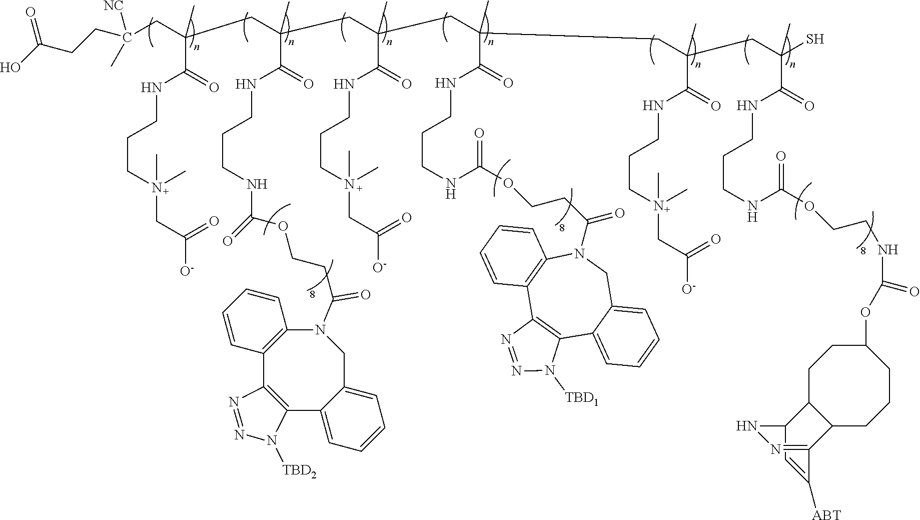
wherein n is an integer between 1 and 50,
or a pharmaceutically acceptable salt or solvate thereof.

14. A method of treating cancer in a subject, the method comprising administering an effective amount of a compound to the subject, wherein the compound comprises:
a plurality of target binding domains (TBDs) that bind to two or more different target proteins expressed by the cancer;
at least one antibody binding domain (ABD) and/or cytotoxic agent; and
a polymer scaffold connecting the plurality of TBDs with the at least one ABD and/or cytotoxic agent.

15. The method of claim 14, wherein the target proteins comprise two or more of PSMA, uPAR, HER2, EGFR, CD38, BCMA and integrins.

16. The method of claim 14, wherein the TBDs comprise: 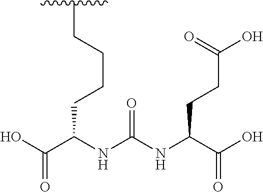 [SMILES: *CCCC[C@H](NC(=O)N[C@@H](CCC(=O)O)C(=O)O)C(=O)O]
to target PSMA; 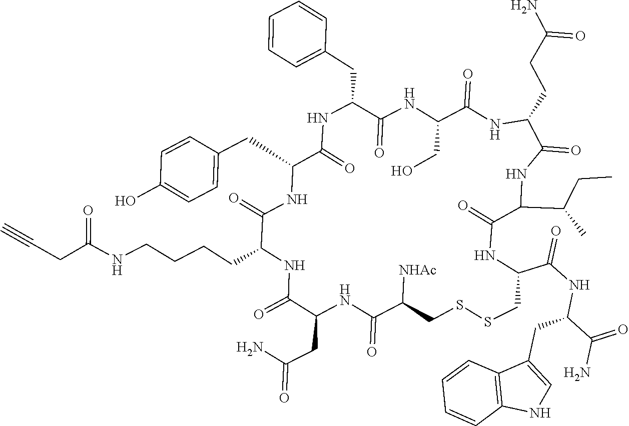 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]
or azido-K-G-S-G-G-D-Cha-F-s-r-Y-L-W-S to target uPAR, wherein Cha is cyclohexylalanine, L-amino acids are shown in upper case and D-amino acids are shown in lower case; and/or 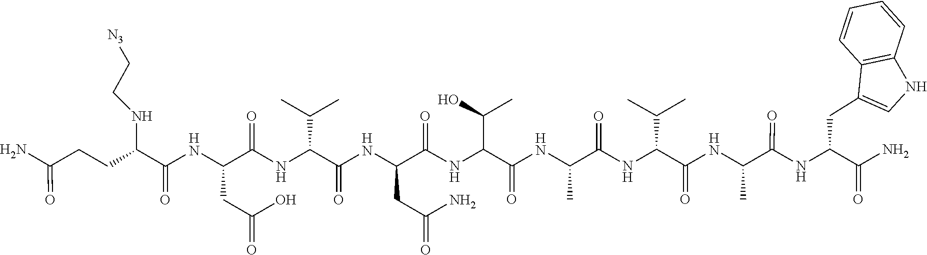 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]
to target HER2.

17. The method of claim 14, wherein the cancer is breast cancer, multiple myeloma, prostate cancer, or glioblastoma.

18. The method of claim 14, wherein:
the cancer is prostate cancer and the target proteins comprise PSMA and HER2;
the cancer is prostate cancer and the target proteins comprise PSMA, HER2 and uPAR;
the cancer is glioblastoma and the target proteins comprise two or more of HER2, uPAR, PSMA and EGFR;
the cancer is multiple myeloma and the target proteins comprise CD38 and BCMA; or
wherein the cancer is breast cancer and the target proteins comprise two or more of HER2, uPAR and EGFR.

19. The method of claim 14, wherein
the ABD is a pan IgG binding ligand;
the cytotoxic agent is doxorubicin, a topoisomerase inhibitor, or a mitotic inhibitor;
the polymer scaffold comprises a ROMP block co-polymer, a RAFT PCB/methyl acrylate co-polymer or a synthetic nucleic acid biopolymer;
the ratio of TBDs: ABDs is between 1:10 and 10:1; and/or
the plurality of TBDs comprises a first TBD (TBD1) and a second TBD (TBD2), wherein
the ratio of TBD1: TBD2 is between 1:10 and 10:1.

20. The compound of claim 19, wherein the compound is: 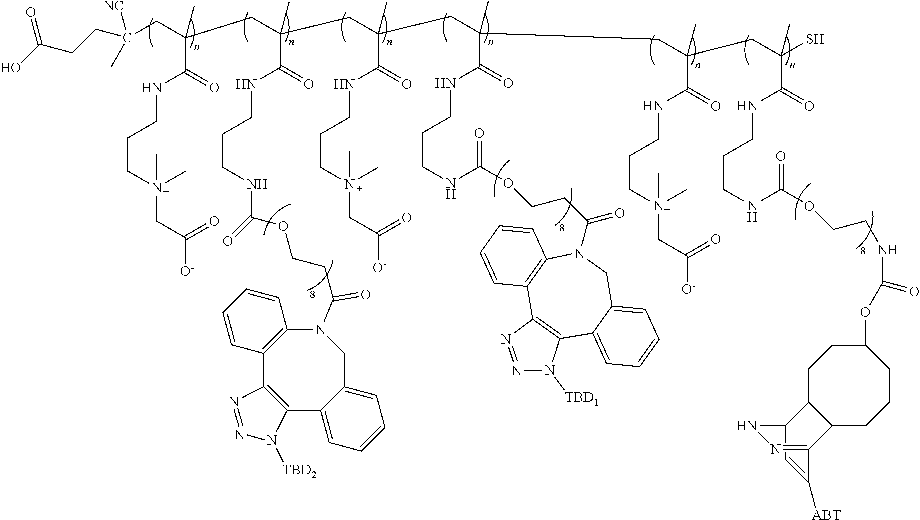
wherein n is an integer between 1 and 50,
or a pharmaceutically acceptable salt or solvate thereof.
